(19) United States
(12) Patent Application Publication
(10) Pub. No.: US 2026/0345996 A1
(43) Pub. Date: Nov. 5, 2026

(54) THERAPEUTIC INSOLE WITH INTEGRATED ELECTRICAL STIMULATION

(71) Applicants: Arul Verma, Warren, MI (US); Aden Wilgus, Kiln, MS (US); Jonathan Tierney, Sherborn, MA (US); Daniel Reynolds, West Baden Springs, IN (US); Shuban Bhatcar, Troy, MI (US)

(72) Inventors: Arul Verma, Warren, MI (US); Aden Wilgus, Kiln, MS (US); Jonathan Tierney, Sherborn, MA (US); Daniel Reynolds, West Baden Springs, IN (US); Shuban Bhatcar, Troy, MI (US)

(21) Appl. No.: 19/665,757

(22) Filed: May 1, 2026

Related U.S. Application Data
(60) Provisional application No. 63/798,241, filed on May 1, 2025.

(51) Int. Cl. A61N 1/04; A43B 3/35; A43B 17/14; A61N 1/02; A61N 1/36

(52) U.S. Cl. CPC A61N 1/0456; A43B 3/35; A43B 17/14; A61N 1/025; A61N 1/0476; A61N 1/36034

(57) ABSTRACT

Examples relate to a therapeutic insole configured to be inserted into footwear. The insole comprises a base structure defining a plantar support surface shaped to conform to an underside of a human foot and a multi-layer body disposed on the base structure comprising a support layer, a cushioning layer, and a surface layer. At least two stimulation electrodes are embedded within the multi-layer body, a first electrode at an arch region and a second electrode at a heel region, accessible at or through the surface layer. An electronics enclosure integrated into the base structure at a heel portion houses a stimulation circuit comprising a microcontroller and one or more power sources. One or more wire routing channels formed within the multi-layer body extend from the electronics enclosure to each stimulation electrode. The stimulation circuit delivers transcutaneous electrical nerve stimulation through the stimulation electrodes.

### CROSS-REFERENCE TO RELATED APPLICATIONS

This application claims the benefit of U.S. Provisional Patent Application Ser. No. 63/798,241, filed May 1, 2025, the entire disclosure of which is incorporated herein by reference.

### TECHNICAL FIELD

Present disclosures relate to therapeutic footwear inserts, particularly therapeutic insoles with integrated electrical stimulation components for foot health and comfort.

### BACKGROUND

Foot health and proper alignment are critical for maintaining mobility and reducing pain in the lower extremities. Conditions such as pes planus, commonly known as flat feet, often lead to discomfort during walking or standing. Traditional solutions often involve the use of orthotic inserts designed to provide mechanical support to the arch of the foot.

While structural support can alleviate some symptoms, passive orthotics are limited in their ability to actively treat underlying muscle fatigue or nerve-related pain. Some existing therapeutic devices utilize electrical stimulation to provide relief, but these are frequently bulky, require external wiring, or are not designed for continuous use within standard footwear during daily activities.

There remains a need for a wearable therapeutic solution that combines structural support with integrated, active electrical stimulation. It would be desirable to provide a low-profile insole capable of delivering targeted therapy while maintaining the comfort and flexibility required for integration into various types of footwear.

### BRIEF SUMMARY

In accordance with aspects of the present disclosure, a therapeutic insole includes a base structure defining a support surface shaped to conform to the underside of a human foot. A multi-layer body is disposed on the base and includes a support layer, a cushioning layer, and a surface layer. Stimulation electrodes are embedded within this multi-layer body at locations corresponding to the arch and heel regions of the foot. An electronics enclosure integrated into the heel portion of the base structure houses a stimulation circuit and power sources, which are connected to the electrodes via routing channels to deliver electrical stimulation. As used herein, electrical stimulation includes, but is not limited to, transcutaneous electrical nerve stimulation (TENS), neuromuscular electrical stimulation (NMES), pulsed electrical stimulation, and other forms of electrical stimulation. In one embodiment, the system delivers transcutaneous electrical nerve stimulation (TENS).

According to some examples, a method of manufacturing a therapeutic insole involves forming a flexible polymer base structure with an enclosure cavity and assembling a multi-layer body thereon. Wire routing channels are formed within a molded elastomeric support layer to facilitate the connection of stimulation electrodes. The process includes positioning the electrodes for access through a surface layer and installing a stimulation circuit within the enclosure to provide electrical therapy through a wiring harness.

In some implementations, a therapeutic stimulation system includes the therapeutic insole and an external device. The insole includes a wireless communication module within its electronics enclosure, allowing it to receive stimulation control signals from a software application executing on the external device. The stimulation circuit is configured to generate and deliver pulsed electrical stimulation signals through the embedded electrodes in response to the signals received from the external device, enabling a coordinated and customizable therapeutic experience.

### BRIEF DESCRIPTION OF THE DRAWINGS

FIG. 1 illustrates a block diagram of a connected neurostimulation system, according to some embodiments of the present disclosure.

FIG. 2 is a top plan view of a smart insole, according to some embodiments of the present disclosure.

FIG. 3 is an exploded view of a multi-layered insole structure, according to some embodiments of the present disclosure.

FIG. 4 illustrates a block diagram of a rigid electronics housing and its internal components, according to some embodiments of the present disclosure.

FIG. 5 is a block diagram of a stimulation circuit, according to some embodiments of the present disclosure.

FIG. 6 is a block diagram of a biphasic symmetric stimulation waveform, according to some embodiments of the present disclosure.

FIG. 7 illustrates a block diagram of a pressure sensor array distribution, according to some embodiments of the present disclosure.

FIG. 8 illustrates a block diagram of a conductive foot covering for electrical stimulation, according to some embodiments of the present disclosure.

FIG. 9 is a flowchart of a method of manufacturing the smart insole, according to some embodiments of the present disclosure.

FIG. 10 illustrates a user interface of a mobile application, according to some embodiments of the present disclosure.

FIG. 11 is a block diagram of a generic computing device architecture, according to some embodiments of the present disclosure.

FIG. 12 is a flowchart of a method of operating the therapeutic insole during a therapy session, according to some embodiments of the present disclosure.

### DETAILED DESCRIPTION

The present disclosure relates generally to the field of wearable therapeutic devices and, more particularly, to foot-worn inserts designed to provide both mechanical support and active neuromuscular relief. In the field of podiatry and orthotics, maintaining proper foot alignment and managing chronic pain are critical for mobility and overall quality of life.

While traditional footwear inserts focus primarily on the passive redistribution of pressure and the structural reinforcement of the foot's natural arches, the present disclosure provides a sophisticated integration of active electronics within a multi-layered orthopedic structure.

This integrated approach allows for the delivery of targeted electrical stimulation directly to the plantar surface of the foot while the user is engaged in daily activities. By combining the structural benefits of a contoured insole with the neurological benefits of electrical stimulation (such as transcutaneous electrical nerve stimulation (TENS), neuromuscular electrical stimulation (NMES), or pulsed electrical stimulation), the disclosed system and method provide a comprehensive solution for managing conditions such as pes planus, commonly known as flat feet, and other forms of chronic foot discomfort.

In some existing approaches to foot pain management, users often rely on rigid or semi-rigid orthotic inserts. While these devices can assist in correcting biomechanical misalignments, they generally do not address the active pain signals transmitted from the nerves in the foot to the brain. In many cases, individuals with diminished or absent foot arches experience significant pain that spreads through the ankle and upper foot due to increased pressure on the ankle bone. Traditional passive inserts may fail to alleviate this sharp pain, leading users to seek external medical treatments or portable electrical stimulation units. However, conventional electrical stimulation devices are typically cumbersome, requiring external wires, adhesive electrode pads, and separate control units that are difficult to use discreetly or comfortably within standard footwear. Furthermore, some existing stimulation techniques may present risks of skin irritation or electrochemical burns if the electrical current is not carefully managed, particularly in the high-friction, high-moisture environment of a shoe. There remains a need for a therapeutic solution that provides seamless, integrated stimulation that is both safe for long-term skin contact and effective at blocking pain signals at their source.

The present disclosure addresses these and other challenges by providing a therapeutic insole with integrated electrical stimulation components. In at least some embodiments, the disclosed system utilizes a multi-layer body disposed upon a specialized base structure. The base structure may be formed from a variety of flexible polymer materials, such as polyethylene terephthalate glycol (PETG) or thermoplastic polyurethane, which provide the necessary structural integrity to conform to the underside of a human foot while remaining flexible enough to accommodate the natural gait cycle. This base structure defines a plantar support surface that mirrors the anatomical contours of the foot, ensuring that the stimulation components remain in consistent contact with the skin. Within this structure, an electronics enclosure is integrated, typically at the heel portion where the material volume is greatest and the impact forces can be managed. This enclosure houses a stimulation circuit, which may include a microcontroller, power sources, and communication modules, all protected from the mechanical stresses of walking.

In some examples, the multi-layer body comprises a series of specialized materials, including a support layer, a cushioning layer, and a surface layer. The support layer may consist of a molded elastomeric material designed to provide a foundation for internal components, while the cushioning layer may utilize high-performance foams, such as ethylene-vinyl acetate or open-cell polyurethane foams, to enhance user comfort and impact absorption. Embedded within these layers are at least two stimulation electrodes, strategically positioned to correspond to the arch region and the heel region of the foot. These electrodes are accessible at or through the surface layer, allowing for direct or near-direct contact with the user's skin. To ensure the safety and comfort of the user, the stimulation circuit is configured to deliver a specific type of electrical signal, such as a biphasic symmetric stimulation waveform. This waveform helps to prevent the buildup of ions at the skin-electrode interface, thereby reducing the risk of skin burns or irritation that can occur with monophasic signals.

According to some implementations, the present disclosure incorporates advanced sensing and environmental control features to optimize the therapeutic experience. For instance, the system may include one or more pressure sensors positioned within the insole structure. These sensors are configured to detect changes in force distribution, allowing the microcontroller to distinguish between a stationary state, such as sitting or standing, and an ambulatory state, such as walking or running. In some examples, the stimulation circuit may be programmed to deliver electrical pulses only when the user is substantially stationary, ensuring that the therapeutic effect is not disrupted by the mechanical noise of movement and that the user can focus on the relief provided. Additionally, some embodiments may include a low-power heating element. This heater can be used to induce a controlled amount of localized perspiration, which increases the moisture at the skin-electrode interface. This increased moisture improves electrical conductivity, ensuring that the stimulation signals effectively penetrate the stratum corneum to reach the underlying nerves without requiring the application of external conductive gels.

The disclosed system further extends its utility through connectivity and intelligent control. In at least one embodiment, the stimulation circuit includes a wireless communication module that allows the insole to interface with an external device, such as a smartphone or a dedicated controller. A software application executing on the external device enables the user to customize the stimulation parameters, including intensity, duration, and frequency, based on their specific pain levels and comfort preferences. This digital integration allows for the collection of usage data and the monitoring of foot health over time. Furthermore, the manufacturing of such a device involves a specialized process of forming wire routing channels within the molded layers to ensure that the internal wiring harness does not create pressure points or discomfort for the user. By routing the electrical connections from the heel-mounted electronics to the arch and heel electrodes through these internal channels, the insole maintains a smooth, anatomical profile.

In summary, the present disclosure provides a sophisticated therapeutic tool that bridges the gap between traditional orthotics and modern neurostimulation technology. By integrating stimulation electrodes and control circuitry directly into a comfortable, multi-layered insole, the disclosed technology offers a discreet and effective means of managing foot pain. The use of safety-conscious waveforms, pressure-sensitive operation, and conductivity-enhancing thermal elements ensures that the device is both effective and comfortable for daily use. The system not only provides the mechanical support necessary for those with foot deformities but also actively engages the body's natural pain-relief mechanisms, such as the release of endorphins and the blocking of pain signals to the brain.

This holistic approach represents a significant advancement in footwear technology, offering users a more comfortable and active movement experience.

The following description provides a detailed overview of the various components and configurations of the disclosed system and method. While specific materials and arrangements may be described in relation to certain examples, it should be understood that the principles of the present disclosure may be applied in various ways across different footwear types and therapeutic applications. The embodiments described herein are intended to be illustrative rather than limiting of the scope of the technology.

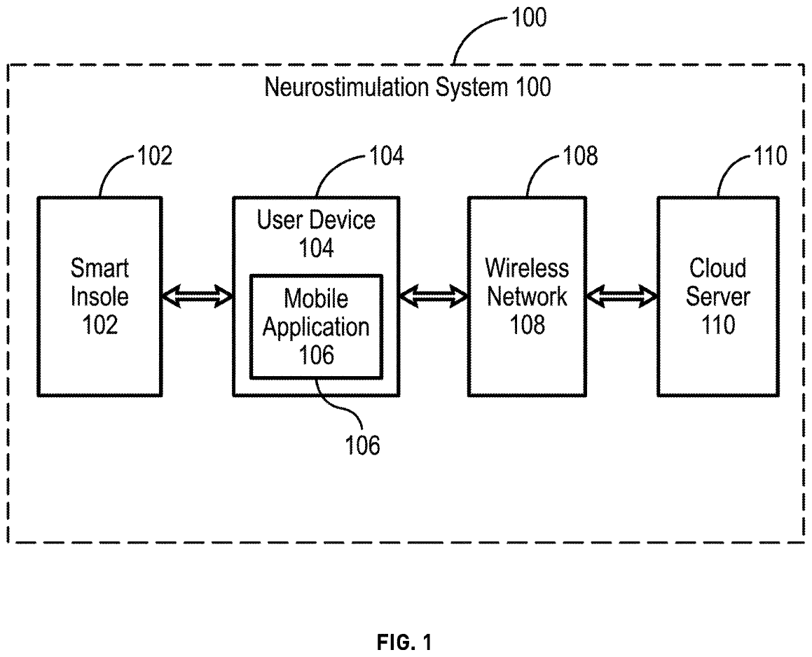

### FIG. 1: Neurostimulation System Architecture

FIG. 1 is a system architecture diagram showing a top-level view of a connected neurostimulation system 100, according to some examples.

A neurostimulation system 100 includes a smart insole 102 configured to communicate with a user device 104. The smart insole 102, described in greater detail with reference to subsequent figures, serves as the primary therapeutic interface of the neurostimulation system 100, delivering targeted electrical stimulation to a user's foot while collecting biomechanical data. By isolating the physical stimulation hardware within the smart insole 102 and offloading complex processing and user interface functions to the user device 104, the neurostimulation system 100 maintains a modular nature. This modularity reduces the computational burden, physical size, and power requirements of the wearable component, allowing the smart insole 102 to remain unobtrusive during daily use.

The user device 104 may include a smartphone, tablet, smartwatch, wearable computer, or dedicated medical controller. A mobile application 106 executes on the user device 104 to provide the user with control over stimulation parameters. At a high level, the mobile application 106 serves as the control hub for the patient. More specifically, the mobile application 106 provides a graphical user interface that allows a user to adjust therapy settings, select stimulation modes, and view real-time or historical foot pressure data. In some examples, the mobile application 106 allows fine-grained manipulation of specific stimulation parameters, such as pulse amplitude, frequency, and duration. Upon receiving user input, the mobile application 106 translates these adjustments into command signals that the user device 104 transmits to the smart insole 102 via a short-range wireless communication link, such as a Bluetooth Low Energy (BLE) or similar personal area network connection.

In addition to local control, the neurostimulation system 100 facilitates wireless data transmission for logging usage and pressure data. The smart insole 102 continuously or periodically transmits telemetry, including stimulation usage logs, battery status, and pressure sensor data, to the user device 104. The user device 104, in turn, routes this collected data through a wireless network 108 to a cloud server 110. The wireless network 108 may include cellular networks, Wi-Fi, or other wide-area network infrastructures. The connection between the wireless network 108 and the cloud server 110 utilizes standard internet or cloud links to ensure secure, encrypted data transfer.

The cloud server 110 provides backend processing and remote monitoring capabilities for the neurostimulation system 100. By receiving the wireless data transmission from the user device 104, the cloud server 110 can aggregate historical usage logs and pressure matrices. This enables longitudinal analysis of patient compliance, therapeutic efficacy, and disease progression. In some examples, healthcare providers can access the cloud server 110 to remotely monitor the patient's status and push updated prescription parameters back down through the wireless network 108 and the mobile application 106 to the smart insole 102.

Those skilled in the art will recognize that modifications and variations are possible without departing from the scope of the present disclosure.

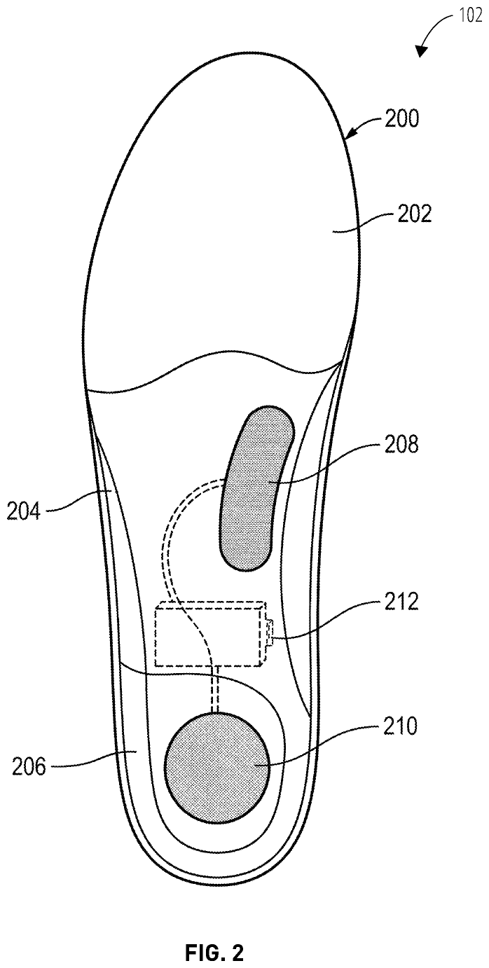

Following the system-level overview of FIG. 1, FIG. 2 provides a closer look at the physical arrangement of the therapeutic components.

### FIG. 2: Smart Insole Top View

FIG. 2 is a top plan view showing a spatial arrangement of a smart insole 102, according to some examples.

The smart insole 102 comprises an insole body 200 that defines the device's overall footprint and structural foundation. The insole body 200 is conceptually and anatomically divided into three primary zones: a forefoot region 202, an arch support region 204, and a heel region 206. The arch support region 204 is characterized by a raised medial contour that may conform to the user's plantar anatomy, providing passive structural support while positioning active components in close proximity to target nerve pathways.

Positioned on the top surface of the insole body 200 are conductive interfaces configured to deliver electrical therapy. An arch stimulation pad 208 is positioned medially within the arch support region 204, shaped generally as an elongated oval to align with the plantar fascia and medial plantar nerve pathways. A heel stimulation pad 210 is positioned centrally within the heel region 206, shaped as a circular or rounded-rectangular patch to target the calcaneal nerve branches. The strategic placement of the arch stimulation pad 208 and the heel stimulation pad 210 enables specific anatomical targeting. Rather than solely blocking direct pain signals at the site of discomfort, this targeted stimulation is configured to induce the body to release natural painkillers, such as endorphins, thereby providing temporary relief from conditions such as flat foot pain, where the natural arch is diminished.

In some examples, the arch stimulation pad 208 and the heel stimulation pad 210 may each comprise a multi-electrode array configuration. By subdividing large single electrodes into multiple smaller independent channels, the smart insole 102 can distribute electrical current more evenly across the skin surface. This multi-channel approach prevents localized current hotspots, thereby reducing the risk of skin irritation or burns during prolonged therapeutic sessions.

In further embodiments, one or more of the stimulation pads may be configured in a guard ring electrode arrangement. In a guard ring configuration, a central active electrode is concentrically surrounded by an annular return electrode. During stimulation, a microcontroller drives current through the central active electrode into the underlying tissue while simultaneously maintaining the annular return electrode at a reference potential or at a controlled sink current level. The annular return electrode intercepts laterally spreading current at the periphery of the active electrode and conducts it back through the return path of the stimulation circuit. This concentric geometry confines the delivered current field to a localized volume of tissue directly beneath the central active electrode, reducing lateral current spread into adjacent tissue regions that are not targeted for stimulation. By constraining the stimulation field in this manner, the guard ring configuration reduces the amount of current that reaches unintended nerve pathways or skin regions outside the target zone, which can otherwise cause diffuse or unfocused stimulation sensations. The guard ring return electrode may be fabricated as a conductive ring trace deposited on the same substrate as the central active electrode, or as a separate annular conductive patch positioned concentrically around the active electrode within the cushioning layer or the support layer of the multi-layer body. In some examples, the width and radial spacing of the annular return electrode relative to the central active electrode are selected based on the target penetration depth and the desired lateral confinement of the current field for a given anatomical zone.

In some embodiments, the stimulation pads may be arranged in a tripolar or quadripolar current steering configuration. In a tripolar configuration, three electrodes are positioned in a linear or triangular spatial arrangement within a single stimulation pad region, such as the arch stimulation pad 208 or the heel stimulation pad 210. A microcontroller independently controls the amplitude and timing of the stimulation signal applied to each of the three electrodes. By varying the relative amplitude ratios among the three electrodes, a microcontroller (e.g., the microcontroller 502 described below) can electronically steer the resulting current field along a desired spatial path within the tissue beneath the stimulation pad without physically repositioning the electrodes. For example, driving a higher proportion of current through a medially positioned electrode relative to a laterally positioned electrode shifts the peak current density toward the medial side of the stimulation zone. In a quadripolar configuration, four electrodes are arranged in a rectangular, diamond, or other two-dimensional spatial pattern. The four-electrode arrangement provides two independent axes of current steering, allowing the microcontroller 502 to direct the current field in both the medial-lateral and the anterior-posterior directions within the plane of the stimulation pad. In both tripolar and quadripolar configurations, the microcontroller 502 may execute a current steering algorithm that receives input from the pressure sensors 516, the wireless communication module 504, or a pre-programmed stimulation protocol stored in memory, and adjusts the per-electrode amplitude values to maintain the current field centered on the target nerve pathway as the user's foot shifts position on the insole during use. A current steering algorithm may operate in conjunction with the biphasic symmetric waveform generation described herein, such that each electrode in the tripolar or quadripolar array delivers a charge-balanced biphasic pulse at the amplitude ratio assigned by the steering algorithm.

In some implementations, the microcontroller 502 cycles through a sequence of steering configurations during a single stimulation session to distribute the delivered charge across a broader tissue area, reducing the likelihood of localized tissue fatigue or habituation to the stimulation signal at any single electrode site. Those skilled in the art will recognize that the guard ring, tripolar, and quadripolar electrode configurations described above may be used individually or in combination with each other and with the multi-electrode array configuration, and that the number, geometry, and spatial arrangement of electrodes within each stimulation pad may be varied without departing from the scope of the present disclosure.

In some embodiments, the conductive surfaces of the stimulation pads (e.g., the arch stimulation pad 208 and the heel stimulation pad 210) are coated with a conductive polymer material to improve electrical performance at the electrode-skin interface. One example of a suitable conductive polymer is poly(3,4-ethylenedioxythiophene) polystyrene sulfonate, commonly referred to as PEDOT: PSS. PEDOT: PSS is a solution-processable conductive polymer that can be deposited onto electrode substrates from an aqueous or solvent-based dispersion using techniques such as drop casting, spin coating, screen printing, or dip coating. Following deposition, the PEDOT: PSS layer is subjected to a thermal curing process, which may include baking at temperatures in the range of approximately 80 °C to 150 °C for a duration sufficient to evaporate the carrier solvent and cross-link the polymer matrix. This thermal curing produces a mechanically stable conductive film that adheres to the underlying electrode substrate and resists delamination under the repeated cyclic loading and shear forces generated by the user's foot during standing and ambulation.

The use of PEDOT: PSS as an electrode coating offers several functional advantages for transcutaneous electrical nerve stimulation in footwear. First, PEDOT: PSS exhibits mixed ionic and electronic conductivity, meaning that it can transport both electrons within the polymer backbone and ions at the polymer-electrolyte boundary. This mixed conduction mode reduces the magnitude of the Helmholtz double-layer capacitance that forms at the electrode-electrolyte interface when electrical current is delivered through the skin. In a conventional metallic electrode, the interface between the electrode surface and the electrolytic medium (e.g., sweat or moisture on the skin) behaves as a parallel-plate capacitor, where counter-ions in the electrolyte accumulate opposite to the charge on the electrode surface. This Helmholtz double-layer capacitance introduces a reactive impedance component that opposes the flow of stimulation current, particularly at the onset of each pulse phase, and can cause voltage transients that reduce the efficiency of charge delivery to the target nerve pathways. By coating the electrode with PEDOT: PSS, the effective electrode-electrolyte interface transitions from a purely capacitive surface to a mixed faradaic and capacitive interface, where charge transfer occurs partly through volumetric ion exchange within the polymer film rather than solely through surface capacitive charging. This reduces the interfacial impedance and allows a greater proportion of the applied stimulation energy to be delivered to the underlying tissue rather than being stored as capacitive charge at the electrode surface.

Second, PEDOT: PSS coatings exhibit durability under conditions representative of use within footwear. Testing of PEDOT: PSS films on textile and polymer substrates has demonstrated that the conductive coating can withstand on the order of 50 wash cycles with minimal degradation of electrical conductivity. In the context of the smart insole 102, where the stimulation pads are embedded within a multi-layer body and are not directly laundered but are exposed to moisture from perspiration and mechanical abrasion from the user's foot, this wash-cycle durability indicates that the PEDOT: PSS coating maintains its conductive properties over extended periods of use without requiring reapplication. The biocompatibility of PEDOT: PSS is also favorable for prolonged skin contact applications, as the material does not release cytotoxic byproducts under normal operating conditions.

Third, the solution-processable nature of PEDOT: PSS allows it to be applied during the insole manufacturing process described herein without requiring vacuum deposition, sputtering, or other high-cost thin-film fabrication techniques. The PEDOT: PSS dispersion can be applied to the stimulation pad substrates prior to their embedding within the cushioning layer or the support layer, and the thermal curing step can be integrated into the existing thermal bonding operations used to assemble the multi-layer stack. In alternative embodiments, other conductive polymer coatings, such as polypyrrole or polyaniline, or hybrid coatings combining conductive polymers with carbon nanotube or graphene additives, may be used in place of or in combination with PEDOT: PSS to tailor the electrode impedance characteristics, mechanical flexibility, or environmental resistance for specific application requirements.

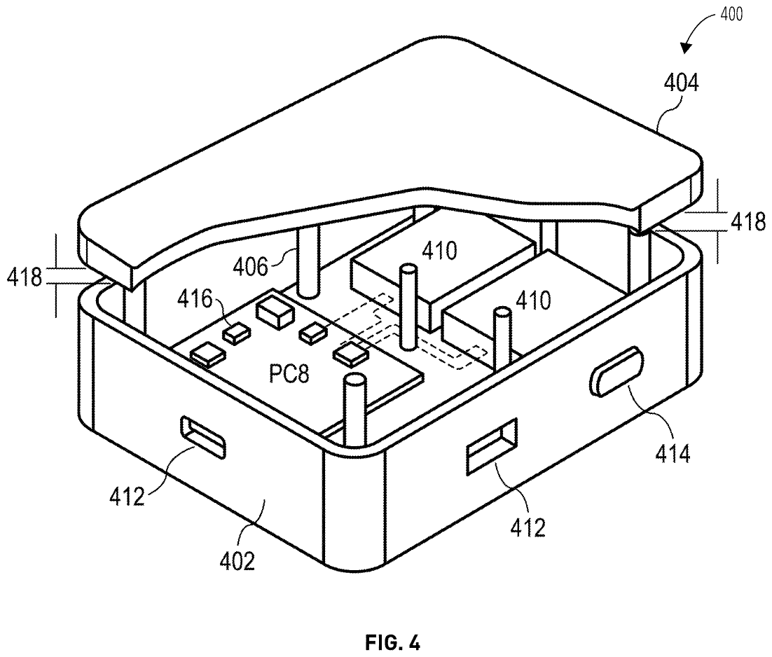

Located beneath the surface of the insole body 200, for example in the transition area between the heel region 206 and the arch support region 204, is a rigid electronics housing 212. The rigid electronics housing 212, which is described in greater detail below with reference to FIG. 4, provides a protective enclosure for the sensitive power and control circuitry required to generate the stimulation waveforms. In the context of the insole body 200, the rigid electronics housing 212 is strategically embedded within the cushioning layers to shield the rigid components from direct impact forces during heel strike while maintaining proximity to the stimulation pads to minimize internal wiring lengths. Internal wiring routes electrical signals from the rigid electronics housing 212 to the respective pads, with a curved path extending through the internal layers to the arch stimulation pad 208 and a shorter, more direct path connecting to the heel stimulation pad 210.

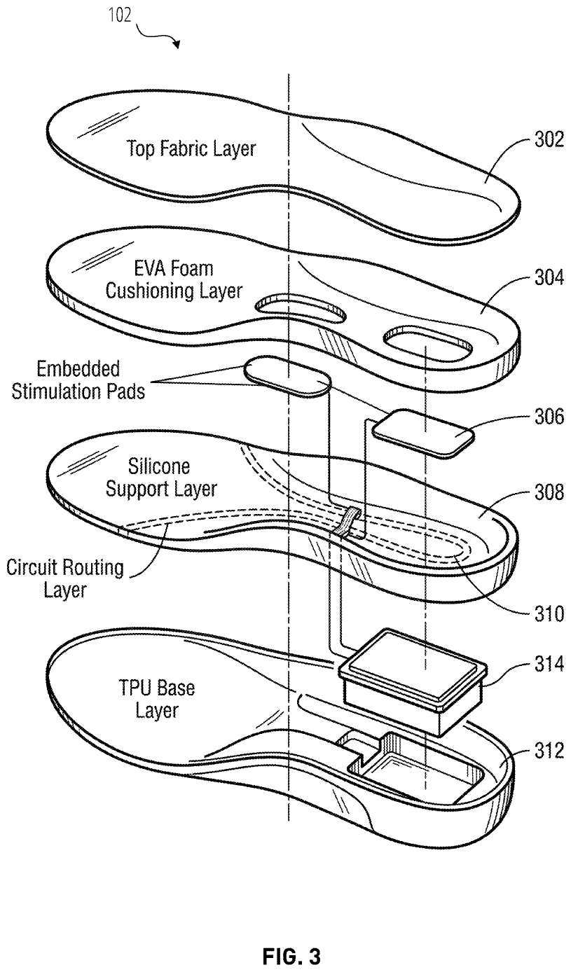

The integration of these surface and internal components relies on a multi-layered structure, which is described in further detail below with reference to FIG. 3. The multi-layered structure provides a balance of mechanical shock absorption, structural rigidity for the electronics, and conductive pathways for the internal wiring. By distributing the components across specialized layers, the insole body 200 maintains a comfortable movement experience for the user while effectively housing the necessary therapeutic hardware.

### FIG. 3: Exploded View of Insole

FIG. 3 is a vertical exploded view showing a layered structural hierarchy of a smart insole 102, according to some examples.

Following the external configurations described in previous figures, the illustrated exploded view reveals the vertical arrangement of materials and the strategic positioning of stimulation and electronic components. At the top of the assembly stack, aligned along a central vertical assembly axis, is a top fabric layer 302, depicted as a contoured thin sheet.

The top fabric layer 302 is configured to directly interface with the plantar surface of a user's foot, providing a breathable and moisture-wicking contact surface. In some examples, conductive fabric patches may be disposed on the outer face of the top fabric layer 302 at positions corresponding to the arch stimulation pad 208 and the heel stimulation pad 210. The conductive fabric patches may be formed from a moisture-retaining conductive material, such as a textile woven or knitted from silver-carbon fibers, and are configured to provide an electrical conduction path between the underlying stimulation electrodes and the plantar surface of the user's foot. The conductive fabric patches retain moisture at the electrode-skin interface to sustain delivery of electrical stimulation under low-perspiration conditions. The conductive fabric patches are electrically coupled to the stimulation electrodes through apertures or conductive regions in the top fabric layer 302, such that electrical current generated by the stimulation circuit passes from the electrodes, through the conductive fabric patches, and into the user's skin. In some examples, a low-power heating element layer may be integrated immediately adjacent to or within the top fabric layer 302. This heating element layer is operable to induce localized sweating on the user's foot, which advantageously improves skin-to-pad electrical conductivity for subsequent nerve stimulation operations.

Directly beneath the top fabric layer 302 is an EVA foam cushioning layer 304. The EVA foam cushioning layer 304 is configured to absorb impact forces during ambulation and distribute mechanical loads across the foot. In some implementations, specific material pairings are used to optimize the insole's dynamic response; for instance, Poron may be combined with EVA foam in the flexible layers to provide a superior balance of shock absorption and energy return. The EVA foam cushioning layer 304 includes physical cutouts or recesses designed to accommodate embedded stimulation pads 306. The embedded stimulation pads 306, the functional electrical operations of which are described in further detail with reference to subsequent figures, are shown as two distinct thin patches. By placing one patch in an arch region and another in a heel region, the smart insole 102 is configured to deliver targeted electrical stimulation to designated nerve clusters while maintaining a flush, comfortable surface that prevents localized pressure points.

Shown below the embedded stimulation pads 306 and the EVA foam cushioning layer 304 is a silicone support layer 308. The silicone support layer 308 acts as a molded elastomer layer that overlies the rigid base layer and conforms to the arch contour of the base layer, providing a compliant interface between the user's foot and the underlying structural support to enhance user comfort. The underlying rigid base layer, rather than the silicone support layer 308, provides the arch support. Furthermore, the silicone support layer 308 integrates a circuit routing layer 310, represented by dashed paths indicating wiring or thin ribbon cables. The circuit routing layer 310 provides a secure, flexible electrical connection from the base electronics to the embedded stimulation pads 306. By routing the wiring through channels molded directly into or resting on the silicone support layer 308, the internal conductive elements are protected from repetitive mechanical stress, shear forces, and moisture ingress during user movement.

The foundation of the layered assembly is a TPU base layer 312. The TPU base layer 312 functions as a structural shell that stabilizes the entire multi-layer stack. In some examples, the TPU base layer 312 utilizes a combination of polyethylene terephthalate glycol (PETG) in conjunction with thermoplastic polyurethane (TPU) to establish specific rigidity-to-flexibility gradients along the longitudinal axis of the insole. For example, the heel and midfoot sections may feature a higher PETG concentration for rigid structural support, while the forefoot region relies predominantly on TPU to allow natural toe-off flexion. The TPU base layer 312 includes a recessed cavity at the junction of the heel and arch regions, sized and shaped to receive a rigid electronics housing 314. The rigid electronics housing 314, internal components of which are described in detail with reference to FIG. 4, is nested securely within this recessed cavity. This placement ensures the sensitive electronic components are protected by the rigid shell of the TPU base layer 312 while minimizing any perceptible bulk or discomfort for the user.

In some implementations, adjacent layers of the multi-layer stack are joined by an adhesive scheme selected for compatibility with the material of each layer. For example, a silicone primer may be applied to the TPU base layer 312 and a silicone adhesive may be applied to an overlying silicone support layer, such that curing of the silicone adhesive bonds the silicone support layer to the primed TPU base layer 312. A silicone adhesive may further be applied between the silicone support layer and an overlying fabric layer to bond the silicone support layer to the fabric layer, and a silicone adhesive may further be applied to bond one or more conductive patches to the fabric layer. In further implementations, an adhesive selected for compatibility with open-cell or closed-cell polymer foam, such as a polyurethane-based or contact-type foam adhesive, is applied to bond the fabric layer to an adjacent foam cushioning layer, taking advantage of the porous surface of the foam to promote mechanical interlock in addition to chemical adhesion. Those skilled in the art will recognize that the inter-layer bonding measures described above may be used individually or in combination, and that the selection of a particular primer, adhesive, or bonding sequence may be varied based on manufacturing throughput, cure-time constraints, or target delamination resistance of a given implementation without departing from the scope of the present disclosure.

Those skilled in the art will recognize that modifications and variations are possible without departing from the scope of the present disclosure. For instance, while specific material transitions from flexible foam to molded silicone to a rigid TPU base are depicted, alternative polymer blends, composite structures, or differing numbers of layers may be substituted based on specific footwear applications, varying weight distributions, or distinct orthopedic requirements.

### FIG. 4: Rigid Electronics Housing Assembly

FIG. 4 is a perspective detail view showing an internal arrangement of a rigid electronics housing 400, according to some examples.

Following the multi-layer construction detailed in FIG. 3, FIG. 4 illustrates the structural protection mechanism for the embedded electronic systems. The rigid electronics housing 400 is configured to be embedded within an insole body 200, described above with reference to FIG. 2, to protect sensitive components from the extreme compressive forces generated during human locomotion. At a high level, the housing provides a load-bearing structure configured to transfer applied weight away from internal circuitry and power sources, thereby ensuring the continuous operation of a stimulation system.

The foundation of the structure is a housing box 402, depicted as a rectangular enclosure with rounded corners and vertical side walls. A reinforced lid 404 is positioned over the housing box 402, shown partially cut away to reveal the internal components. To prevent structural collapse under the user's weight, a plurality of support pillars 406 are distributed within the enclosure. The support pillars 406 extend vertically from the floor of the housing box 402 to the underside of the reinforced lid 404. In some examples, the reinforced lid 404 acts as a primary load-bearing surface and rests directly on the support pillars 406 and the upper edges of the side walls of the housing box 402. This arrangement establishes a direct structural load path through the reinforced lid 404 and the support pillars 406, entirely bypassing the sensitive internal electronics.

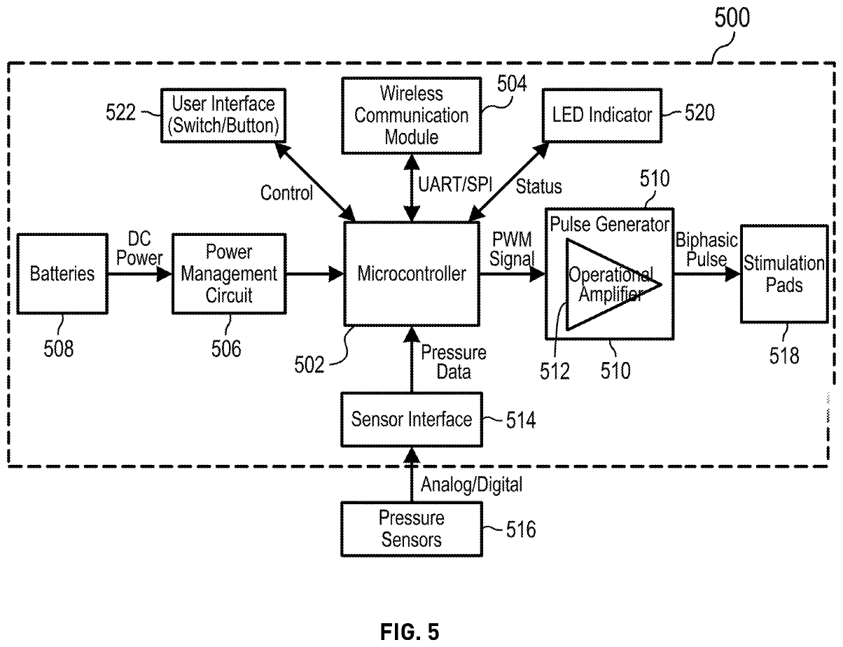

Mounted within the housing box 402 is a printed circuit board (PCB) assembly 408, which occupies a portion of the internal floor space, and a set of batteries 410 positioned side-by-side in the remaining space. The PCB assembly 408 provides a substrate for mounting various electronic components 416. These electronic components 416 may include a microcontroller 502, described below with reference to FIG. 5, configured to manage stimulation waveforms. By routing the structural load path through the support pillars 406, an internal gap 418 is maintained between the underside of the reinforced lid 404 and the top surfaces of both the batteries 410 and the electronic components 416. Maintaining this internal gap 418 is useful for battery safety, as it prevents external compression of the batteries 410 that could otherwise lead to physical deformation, internal short circuits, or thermal runaway.

In some examples, the structural geometry of the support pillars 406 is specifically designed to distribute a maximum expected load across the housing. For instance, the internal pillar geometry and the reinforced lid 404 may be engineered to withstand a static load of at least 1112 Newtons (approximately 250 pound-force) without deflecting into the internal gap 418. To achieve this structural integrity, the reinforced lid 404 and the housing box 402 may be fabricated from high-compressive-strength polymers, such as polyethylene terephthalate glycol (PETG) or polylactic acid (PLA). PETG provides a favorable combination of rigidity and impact resistance for electronics enclosures, while PLA exhibits a flexural modulus in the range of approximately 2,700 to 4,000 MPa, which provides sufficient stiffness to limit deflection of the reinforced lid 404 to less than the dimension of the internal gap 418 under the expected distributed compressive load. In some embodiments, the material for the reinforced lid 404 is selected based on its flexural modulus such that the lid resists deformation into contact with the electronic components 416 and the batteries 410 when the insole is subjected to a compressive load corresponding to a body weight of a user. In some embodiments, the housing components may be constructed from polyethylene terephthalate glycol (PETG), thermoplastic polyurethane (TPU), polycarbonate, or carbon-fiber-reinforced composites. Furthermore, while discrete cylindrical or block-like support pillars 406 are shown, the internal load-bearing architecture may comprise a honeycomb lattice, intersecting ribbed walls, or a solid matrix with customized internal cavities for the electronics.

The electronic architecture within the housing box 402 may feature a dual-battery configuration. Power wiring connects the terminals of the two batteries 410 to the PCB assembly 408. In some implementations, this dual-battery circuit architecture is configured to provide redundant power and load balancing for the stimulation system. By using two separate cells, the system can distribute current during high-intensity stimulation phases, thereby reducing thermal stress on individual cells and extending the overall operational lifespan of the power source.

A user interface and external connections are integrated into the side walls of the housing box 402. In some embodiments, a charging port 412 is provided as an aperture in the side wall, enabling an external power source, such as a Universal Serial Bus (USB) cable connected to a wall adapter or portable battery pack, to deliver charging current to the batteries 410 through a charging circuit mounted on the PCB assembly 408. The charging circuit may include a charge controller integrated circuit configured to regulate the charging voltage and current applied to the batteries 410 in accordance with the battery chemistry (e.g., a constant-current/constant-voltage charging profile for lithium-polymer cells) and to terminate charging upon reaching a full-charge voltage threshold. In alternative embodiments, the charging port 412 may be omitted, and the batteries 410 may be configured as replaceable cells that a user removes from the housing box 402 for external charging or replacement with fresh cells.

In some embodiments, the charging port 412 may be replaced or supplemented by a wireless inductive charging coil mounted within or on the housing box 402, the inductive charging coil being configured to receive electromagnetic energy from a corresponding transmitter coil in an external charging pad and to convert the received energy into direct current for charging the batteries 410. Additionally, a switch 414 is mounted on the side wall to provide a hardware toggle or button for user control, such as powering the device on or off. In some embodiments, the switch 414 is a multi-function switch configured to accept distinct input patterns, such as a single press to toggle power on or off, a double press to initiate a wireless pairing mode for the wireless communication module 504, and a press-and-hold action to enter a factory reset or diagnostic mode. The microcontroller 502 detects the input pattern applied to the switch 414 and transitions the device to the corresponding operational state.

In some embodiments, one or more light-emitting diode (LED) indicators are mounted on the PCB assembly 408 and are visible through an aperture, a light pipe, or a translucent window formed in the side wall of the housing box 402. The one or more LED indicators are driven by a microcontroller to convey device status information to the user. For example, a first LED indicator may emit a steady illumination in a first color (e.g., green) to indicate that the device is powered on and operating, and may emit no light when the device is powered off. A second LED indicator, or the same LED indicator driven in a different color or blink pattern, may convey battery charge status. For instance, the LED indicator may emit a steady illumination in a second color (e.g., blue) when the battery charge level is above a predetermined threshold (e.g., above 20% of full capacity), may emit a blinking pattern in a third color (e.g., red) when the battery charge level falls below the predetermined threshold to alert the user that the batteries 410 require charging or replacement, and may emit a pulsing or breathing pattern in a fourth color (e.g., amber) when the batteries 410 are actively being charged via the charging port 412 or the wireless inductive charging coil. A further LED indicator, or a further blink pattern of the same LED indicator, may convey wireless connection status. For example, the LED indicator may emit a rapid blinking pattern to indicate that the wireless communication module 504 is in pairing mode and awaiting a connection request from the user device 104, and may emit a steady illumination or a slow periodic blink to indicate that a wireless connection has been established with the user device 104. In some implementations, the microcontroller 502 drives the one or more LED indicators using pulse-width modulation to control brightness and to produce the blinking, pulsing, or breathing patterns described above. In alternative embodiments, the LED indicators may be replaced by or supplemented with a small organic light-emitting diode (OLED) display or an electronic ink (e-ink) display panel mounted on or visible through the side wall of the housing box 402, the display panel being configured to render alphanumeric or graphical status information, such as a battery percentage, a stimulation intensity level, a wireless connection icon, or a fault code.

In some embodiments, the PCB assembly 408 may comprise a flexible printed circuit board (also referred to as a flex PCB or flexible printed circuit) in place of a rigid circuit board substrate. A flexible printed circuit board utilizes a thin polyimide or polyester film as the dielectric substrate, with conductive copper traces laminated or deposited onto the film.

The polyimide substrate is bendable and can conform to curved or non-planar surfaces within the housing box 402 or the insole body 200 more readily than a rigid FR-4 or similar glass-reinforced epoxy laminate substrate. By replacing the rigid PCB with a flexible printed circuit board, the vertical profile of the PCB assembly 408 within the housing box 402 can be reduced, because the flexible substrate can be shaped to follow the contour of the floor of the housing box 402 rather than requiring a flat, planar mounting surface. This reduced profile allows the overall height of the housing box 402 and the reinforced lid 404 to be decreased, which in turn reduces the perceptible bulk of the electronics enclosure within the insole body 200.

Additionally, a flexible printed circuit board distributes mechanical strain across a larger area of the substrate when the insole is subjected to repeated bending during the gait cycle, rather than concentrating stress at discrete solder joints as can occur with a rigid board mounted within a flexing structure. This strain distribution reduces the likelihood of solder joint fatigue failures and trace fractures over the operational lifetime of the insole.

In some implementations, the flexible printed circuit board may be a rigid-flex hybrid construction, in which rigid board sections carrying components such as the microcontroller 502, the operational amplifier 512, and battery connector clips are joined by flexible polyimide bridge sections. The rigid sections provide stable mounting surfaces for surface-mount components, while the flexible bridge sections accommodate bending between the rigid sections and allow the overall assembly to conform to the internal geometry of the housing box 402.

In further implementations, the flexible printed circuit board may extend beyond the housing box 402 and serve as the circuit routing layer 310 described above with reference to FIG. 3, replacing or supplementing discrete wiring harnesses with integrated flexible conductive traces that route directly from the electronics enclosure to the stimulation pads.

Those skilled in the art will recognize that modifications and variations are possible without departing from the scope of the present disclosure. For example, the charging port 412 may be replaced or supplemented by a wireless inductive charging coil, and the mechanical switch 414 may be replaced by a power button, a capacitive touch sensor, or an entirely software-driven power management state triggered by motion sensors.

In some embodiments, the rigid electronics housing 400 and the PCB assembly 408 incorporate one or more moisture protection measures to resist the ingress of perspiration and ambient humidity generated within footwear during use. For example, a conformal coating may be applied over the populated surfaces of the PCB assembly 408 following component placement and soldering. The conformal coating may comprise an acrylic, silicone, urethane, or parylene film deposited by spraying, dipping, or vapor deposition, forming a continuous dielectric barrier that covers exposed conductive traces, solder joints, and component leads while permitting heat dissipation from active components.

In some implementations, a compressible gasket or O-ring seal is positioned between the reinforced lid 404 and the upper rim of the housing box 402 to form a peripheral moisture barrier when the lid is secured to the housing box 402. The gasket may be formed from silicone rubber, ethylene propylene diene monomer (EPDM), or thermoplastic elastomer and may be seated within a channel molded into the rim of the housing box 402 or the underside perimeter of the reinforced lid 404. In further implementations, voids within the housing box 402 surrounding the PCB assembly 408 and the batteries 410 may be filled, in whole or in part, with an electrically insulating potting compound, such as a two-part epoxy or silicone encapsulant, that cures to form a solid or semi-rigid mass encapsulating the electronic components and their interconnections. The potting compound prevents moisture from reaching solder joints and conductive surfaces and also provides additional mechanical damping against impact and vibration. In alternative or supplementary embodiments, exposed metallic surfaces of the housing box 402, the reinforced lid 404, or the charging port 412 aperture may be treated with a hydrophobic nano-coating, such as a fluoropolymer or organosilane surface treatment, that causes liquid water and perspiration to bead and shed from the treated surface rather than wicking into joints or openings. Those skilled in the art will recognize that the moisture protection measures described above may be used individually or in combination, and that the selection of a particular sealing, coating, or potting approach may be varied based on the target ingress protection rating, manufacturing cost constraints, or serviceability requirements of a given implementation without departing from the scope of the present disclosure.

### FIG. 5: Nerve Stimulation Circuit Diagram

FIG. 5 is a block diagram showing a functional view of a nerve stimulation circuit 500, according to some examples.

The nerve stimulation circuit 500 provides the electrical architecture for generating and regulating therapeutic electrical pulses. At a high level, the nerve stimulation circuit 500 includes a microcontroller 502 configured to execute control logic, process sensor feedback, and dictate stimulation parameters. The microcontroller 502 is operably coupled to a power management circuit 506, which receives direct current (DC) power from one or more batteries 508 and provides a regulated supply voltage (Vcc) to the microcontroller 502 and other active components.

In some embodiments, the power management circuit 506 manages power distribution between dual-battery cells to optimize runtime. In some embodiments, the power management circuit 506 includes an overcurrent protection device, such as a resettable positive temperature coefficient (PTC) fuse, positioned in the power supply path between the one or more batteries 508 and the downstream stimulation circuit components. The PTC fuse is configured to increase in resistance and interrupt current flow when the current through the power supply path exceeds a predetermined threshold, such as in the event of a short circuit or other overcurrent condition. Upon removal of the overcurrent condition and subsequent cooling of the PTC element, the fuse resets to its low-resistance state, restoring normal circuit operation without requiring component replacement. To conserve energy, the microcontroller 502 and the power management circuit 506 may implement low-power state transitions, selectively powering down peripheral modules such as wireless transceivers or high-voltage generation stages during periods of inactivity or when the device is not actively delivering stimulation.

To enable external control and data logging, a wireless communication module 504 communicates with the microcontroller 502 via standard serial protocols, such as Universal Asynchronous Receiver-Transmitter (UART) or Serial Peripheral Interface (SPI). In some implementations, the wireless communication module 504 comprises a Bluetooth Low Energy (BLE) transceiver configured to receive wireless control signals adjusting stimulation intensity and operational modes. For example, a user may wirelessly toggle between low, high, or automatic stimulation modes via an external device. Local control and status feedback are also provided; a user interface 522, such as a tactile switch (e.g., the switch 414) or button, provides manual control inputs to the microcontroller 502, while an LED indicator 520 receives status signals to visually communicate operational states, battery life, or fault conditions.

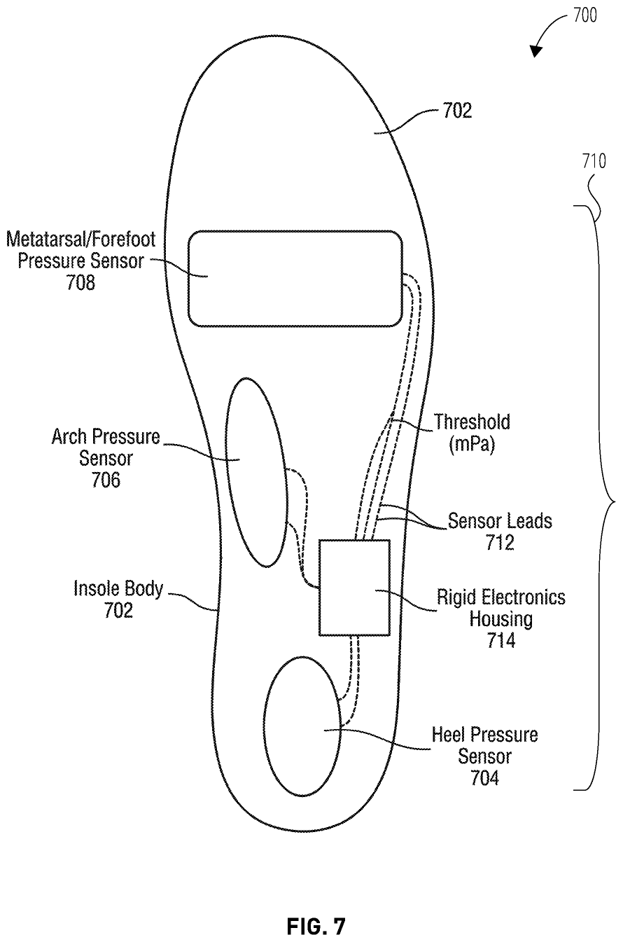

The nerve stimulation circuit 500 includes a sensor interface 514 configured to receive analog or digital signals from pressure sensors 516 and provide corresponding pressure data to the microcontroller 502. The pressure sensors 516, which are described in greater detail with reference to FIG. 7, are used in this circuit to provide real-time mechanical feedback on foot positioning and force distribution. By analyzing the pressure data, the microcontroller 502 may execute gait-based deactivation logic configured to detect a change in force distribution across the plantar surface of the foot. This change corresponds to a transition between a stationary state (e.g., standing or sitting) and an ambulatory state (e.g., walking or running). In some embodiments, the stimulation system is designed for use primarily when the user's foot is in a substantially stationary position relative to the ground.

Accordingly, the microcontroller 502 may automatically suspend or deactivate the pulse generation upon detecting an ambulatory state. To accurately distinguish between subtle gait transitions and transient weight shifts, the sensor interface 514 may utilize specific analog-to-digital converter (ADC) sampling rate parameters, such as sampling the pressure sensors 516 at 50 Hz to 200 Hz, and applying digital low-pass filtering to smooth the pressure data before state evaluation.

For therapeutic delivery, the microcontroller 502 provides a pulse-width modulation (PWM) signal to the pulse generator 510. The pulse generator 510 includes an operational amplifier 512 configured to shape and amplify the PWM signal into a biphasic pulse. In at least one embodiment, the operational amplifier 512 inverts and offsets the unipolar PWM signal received from the microcontroller 502 to produce a biphasic symmetric waveform having positive and negative phases of substantially equal amplitude relative to a zero-baseline reference. This inversion and offset operation causes electrical current to flow in alternating polarity between the stimulation electrodes, ensuring that the electrical stimulation has a net-zero charge transfer to prevent skin burns or tissue damage. The microcontroller 502 dictates the precise PWM control parameters, including the pulse frequency, sample count, and duty cycle, to shape the biphasic waveform. For example, the PWM frequency may be set between 20 Hz and 150 Hz with varying duty cycles to modulate the perceived intensity of the stimulation.

In some embodiments, the pulse generator 510 may be configured to operate in a current-controlled mode rather than a voltage-controlled mode. In the current-controlled mode, the pulse generator 510 includes a current-sensing element, such as a low-value series resistor positioned in the output path between the operational amplifier 512 and the stimulation pads 518. The voltage developed across this sensing resistor is proportional to the instantaneous current flowing through the electrode-skin circuit. The microcontroller 502, or a dedicated feedback network within the operational amplifier 512 circuit, continuously monitors the voltage across the current sensing element and compares it to a target current setpoint stored in the microcontroller 502. When the monitored current deviates from the target setpoint, the feedback loop adjusts the output voltage of the pulse generator 510 to compensate. For example, if skin impedance increases due to drying of the electrode-skin interface, the feedback loop increases the driving voltage to maintain the target current level. Conversely, if skin impedance decreases, such as when perspiration accumulates at the electrode-skin interface, the feedback loop reduces the driving voltage to prevent the delivered current from exceeding the target setpoint. This current-controlled architecture provides consistent stimulation delivery despite fluctuations in skin impedance caused by variations in perspiration, skin dryness, electrode contact area, or changes in contact pressure during use. In some implementations, the microcontroller 502 enforces a maximum voltage compliance limit on the output of the pulse generator 510 to prevent the feedback loop from driving the output voltage to levels that could exceed the safe operating range of the circuit components or cause discomfort to the user. The target current setpoint may be user-adjustable via the wireless communication module 504 and the mobile application 106, or may be set by the microcontroller 502 based on a selected stimulation mode. In further implementations, the microcontroller 502 may log impedance data derived from the ratio of the applied voltage to the measured current, and transmit this impedance data to the external device for display or analysis, providing the user or a healthcare provider with information regarding electrode contact quality over time.

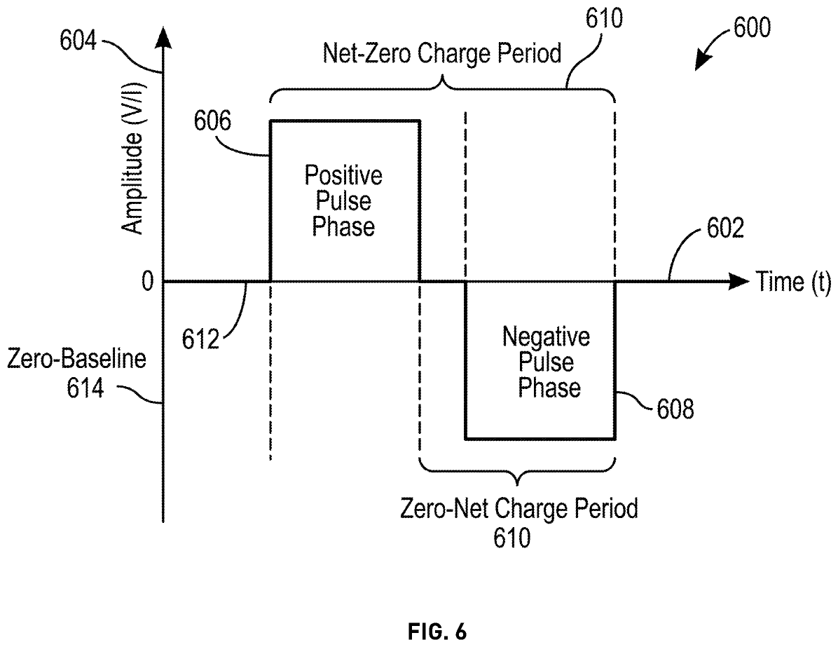

In some embodiments, the nerve stimulation circuit 500 may be configured to generate the biphasic symmetric stimulation waveform using a single battery rather than the dual-battery configuration described above with reference to FIG. 4. In such embodiments, the pulse generator 510 includes an H-bridge switching circuit comprising four metal-oxide-semiconductor field-effect transistors (MOSFETs) arranged in a bridge topology between the single battery and the stimulation pads 518. The microcontroller 502 drives the gate terminals of the four MOSFETs in alternating diagonal pairs to reverse the polarity of the voltage applied across the stimulation pads 518 during each phase of the biphasic waveform. During a first phase, the microcontroller 502 activates a first diagonal pair of MOSFETs, causing current to flow in a first direction through the electrode-skin circuit. During a second phase, the microcontroller 502 deactivates the first diagonal pair and activates a second diagonal pair, reversing the current flow direction to produce the opposite-polarity phase of the biphasic pulse. The timing and duration of MOSFET switching are controlled by the microcontroller 502 to maintain the biphasic symmetry and zero-net charge characteristics described above with reference to FIG. 6.

In some implementations, the single-battery embodiment further includes a DC-DC converter, such as a boost converter or a buck-boost converter, positioned between the single battery and the H-bridge circuit. The DC-DC converter steps up or regulates the output voltage of the single battery cell to a level sufficient to drive the stimulation current through the skin impedance at the electrode-skin interface while remaining within the safe current limits described herein. By using a single battery with an H-bridge and an optional DC-DC converter, the overall volume and weight of the power source within the electronics enclosure can be reduced relative to the dual-battery configuration, which may allow for a thinner profile of the rigid electronics housing 400 or an increase in the available internal volume for other components. Those skilled in the art will recognize that other switching topologies, such as a half-bridge circuit with a split-rail capacitor network, may be substituted for the full H-bridge without departing from the scope of the present disclosure.

The pulse generator 510 outputs the amplified biphasic pulse to stimulation pads 518. The stimulation pads 518, as described above with reference to FIG. 2, receive the generated biphasic waveforms to safely deliver transcutaneous electrical nerve stimulation to targeted areas, such as the arch and heel regions. Furthermore, to ensure electrical coupling between the stimulation pads 518 and the user's skin, the microcontroller 502 may implement heater control logic. In some embodiments, the nerve stimulation circuit 500 controls a low-power heating element situated near the stimulation pads 518. The microcontroller 502 utilizes a feedback loop to maintain a precise temperature threshold (e.g., 37 °C to 40 °C) designed to safely induce localized moisture or sweat without causing user discomfort. This induced moisture significantly lowers skin impedance, thereby improving the electrical stimulation's conductivity and efficacy.

Those skilled in the art will recognize that modifications and variations are possible without departing from the scope of the present disclosure. For instance, alternative waveform topologies, different wireless communication protocols, or additional biometric sensors may be integrated into the nerve stimulation circuit 500 without materially altering the underlying functional architecture.

### FIG. 6: Biphasic Symmetric Waveform

FIG. 6 is a timing diagram showing a temporal view of a biphasic symmetric stimulation waveform 600, according to some examples.

Following the description of the nerve stimulation circuit in FIG. 5, FIG. 6 illustrates the electrical characteristics of the stimulation signal generated by the system. Specifically, the waveform 600 is generated by the pulse generator 510 under the control of the microcontroller 502, both described above with reference to FIG. 5. The microcontroller 502 acts as a central processing unit that defines the timing, frequency, and amplitude parameters, instructing the pulse generator 510 to produce the precisely balanced electrical signal depicted in the diagram.

The diagram shows a horizontal time axis 602 and a vertical amplitude axis 604, which intersect at a zero-baseline 612. The amplitude axis 604 may represent either voltage (V) or current (I), depending on whether the underlying stimulation circuit is configured to operate in a constant-voltage or a constant-current mode. A first positive pulse phase 606 is shown as a rectangular block extending upward from the zero-baseline 612, representing the initial delivery of electrical energy to the target nerve pathways. Following a brief return to the zero-baseline 612 (often referred to as an interphase gap), a negative pulse phase 608 is shown as a rectangular block extending downward from the zero-baseline 612. The transition from the positive peak of the positive pulse phase 606 back to the zero-baseline 612, and subsequently down to the negative peak of the negative pulse phase 608, is executed rapidly to maintain precise temporal control over the stimulation cycle. The negative pulse phase 608 is configured as a mirror image of the positive pulse phase 606 across the time axis 602, having substantially equal amplitude and duration to establish strict biphasic symmetry.

A zero-net charge period 610 is indicated with a bracket spanning both the positive and negative phases, signifying that the total electrical charge delivered during the positive pulse phase 606 is operationally counterbalanced by the charge extracted during the negative pulse phase 608. This charge balancing serves as a physiological safety parameter. Because human skin and underlying tissues act as an electrolytic medium, monophasic or unbalanced biphasic waveforms can cause a net accumulation of ions at the skin-electrode interface. Over time, this electrochemical imbalance drives adverse localized reactions that can lead to skin irritation and, under prolonged exposure to a sustained charge imbalance, to severe chemical burns. By enforcing biphasic symmetry and ensuring a zero-net charge during period 610, the system prevents localized ion buildup and electrochemical tissue damage, thereby enabling safe, continuous neurostimulation on the plantar surface of the foot. In some implementations, safety parameters further include hardware-enforced or software-enforced peak current limits, ensuring the peaks on the amplitude axis 604 never exceed a maximum safe threshold (e.g., capped at 50 mA to 80 mA) regardless of user-selected intensity settings.

In addition to safety, the waveform modulation parameters are specifically tuned to maximize therapeutic efficacy. In various embodiments, the microcontroller 502 dynamically adjusts the pulse width (e.g., spanning durations between 100 to 400 microseconds) and the frequency (e.g., utilizing low-frequency ranges of 1-10 Hz or high-frequency ranges of 50 -150 Hz) of the waveform along the time axis 602. These specific modulation parameters are designed to achieve dual physiological effects: blocking pain signals from reaching the brain via the pain-gate mechanism and stimulating the body to release natural painkillers, such as endorphins. This dual-action approach provides robust temporary relief for localized discomfort, such as the sharp ankle and upper foot pain frequently associated with diminished arches or flat feet.

### FIG. 7: Pressure Sensor Array Distribution

FIG. 7 is a top plan view showing a spatial distribution of a pressure sensing array 700, according to some examples.

FIG. 7 illustrates the structural arrangement and operational logic used to determine when such stimulation is safely applied. An insole body 702, described above with reference to FIG. 2, provides the structural boundary and foundation for the sensing components. A sensor matrix 710 is distributed across the insole body 702. Broadly, the sensor matrix 710 is configured to detect a change in force distribution across a plantar surface of a foot that corresponds to a transition between a stationary state (e.g., standing or sitting) and an ambulatory state (e.g., walking or running).

In some embodiments, the sensor matrix 710 comprises three primary sensing zones corresponding to distinct anatomical regions. A heel pressure sensor 704 is positioned at a posterior end of the insole body 702 to detect heel-strike events. An arch pressure sensor 706 is positioned in a medial region, taking an elongated form to monitor midfoot engagement. A forefoot pressure sensor 708 is positioned in an anterior region, spanning the width of the ball of the foot to detect toe-off forces. Internal sensor leads route along the medial side of the insole body 702, electrically coupling the sensor matrix 710 to an electronics housing 714.

The electronics housing 714, described above with reference to FIG. 4, contains processing components such as a microcontroller 502, described above with reference to FIG. 5.

The microcontroller 502 processes data from the sensor matrix 710 using specific pressure threshold values, typically measured in millipascals (mPa), to classify the user's movement state. By comparing real-time pressure readings against these mPa thresholds, the system executes a binary deactivation logic. If the pressure profile indicates the user is in the stationary state, the system enables electrical stimulation. Conversely, if the pressure profile exceeds the ambulatory thresholds, the system automatically deactivates stimulation. To ensure stable operation, the microcontroller 502 implements debounce logic for the pressure sensor data. This debounce logic prevents rapid, unintended stimulation cycling during the dynamic pressure fluctuations of a standard gait cycle, such as the brief pressure drops between a heel-strike and toe-off. The technical justification for this deactivation logic is to maintain safety and consistent electrode contact; active movement can shift the alignment between the foot and the stimulation pads, and applying stimulation during such misalignment could result in inconsistent delivery of electrical stimulation.

In some embodiments, the microcontroller 502 determines the ambulatory state based on a differential pressure reading between two or more sensor locations within the sensor matrix 710. For example, the microcontroller 502 may compare the pressure signal from the heel pressure sensor 704 against the pressure signal from the forefoot pressure sensor 708 to detect weight transfer patterns characteristic of gait. During a stationary standing state, the pressure distribution between the heel and forefoot remains relatively constant. During walking, the pressure shifts sequentially from the heel to the forefoot as the foot progresses through heel-strike, midstance, and toe-off. The microcontroller 502 detects this sequential weight transfer by monitoring the difference between the heel and forefoot pressure readings over time. When the differential pressure signal exhibits a periodic pattern consistent with a walking cadence, the microcontroller 502 classifies the user as being in the ambulatory state and deactivates the stimulation circuit. This differential pressure approach may be used as an alternative to, or in combination with, the absolute pressure magnitude thresholds described above.

In some implementations, the specific mPa thresholds used by the microcontroller 502 to distinguish the stationary state from the ambulatory state are user-configurable via the mobile application 106 described above with reference to FIG. 1. The mobile application 106 may present a threshold adjustment interface that allows the user to increase or decrease the sensitivity of the ambulatory detection logic based on individual preferences, body weight, or footwear type. The user-configured threshold values are transmitted from the mobile application 106 to the microcontroller 502 via the wireless communication module 504 and stored in non-volatile memory within the microcontroller 502 for use during subsequent operation.

In some embodiments, the microcontroller 502 uses pressure data from the sensor matrix 710 to adjust stimulation parameters, even in the stationary state. For example, while the user is standing, the microcontroller 502 may detect that the pressure profile indicates a concentration of weight on the medial arch region and, in response, increase the stimulation intensity or pulse frequency delivered through the arch stimulation pad 208 to target the nerve pathways in the region experiencing the greatest load. Conversely, if the pressure profile indicates a shift of weight toward the heel, the microcontroller 502 may increase stimulation through the heel stimulation pad 210. This pressure-responsive modulation allows the stimulation circuit to adapt the therapeutic output to the user's real-time weight distribution without requiring manual adjustment by the user or the mobile application 106.

In some implementations, the microcontroller 502 logs pressure sensor data received from the sensor matrix 710 over time and transmits the logged data to the mobile application 106 via the wireless communication module 504. The logged data may include time-stamped records of detected stationary and ambulatory states, cumulative time spent in each state, and the pressure distribution profile during stationary periods. The mobile application 106 may process and display this data to provide the user with feedback regarding the total time spent in stimulation-eligible postures during a given day or session. This usage logging enables the user or a healthcare provider to monitor compliance with a prescribed stimulation regimen and to identify patterns in the user's daily activity that may inform adjustments to the stimulation protocol.

In some embodiments, the sensor matrix 710 may incorporate different sensing technologies, such as a continuous pressure-sensitive film, capacitive arrays, or a higher density matrix of piezoelectric discrete elements. The specific mPa thresholds may be statically pre-programmed or dynamically calibrated to accommodate an individual user's weight, footwear type, and unique gait patterns. Furthermore, the debounce timing windows may be adjustable based on the detected cadence of the user. Those skilled in the art will recognize that modifications and variations are possible without departing from the scope of the present disclosure.

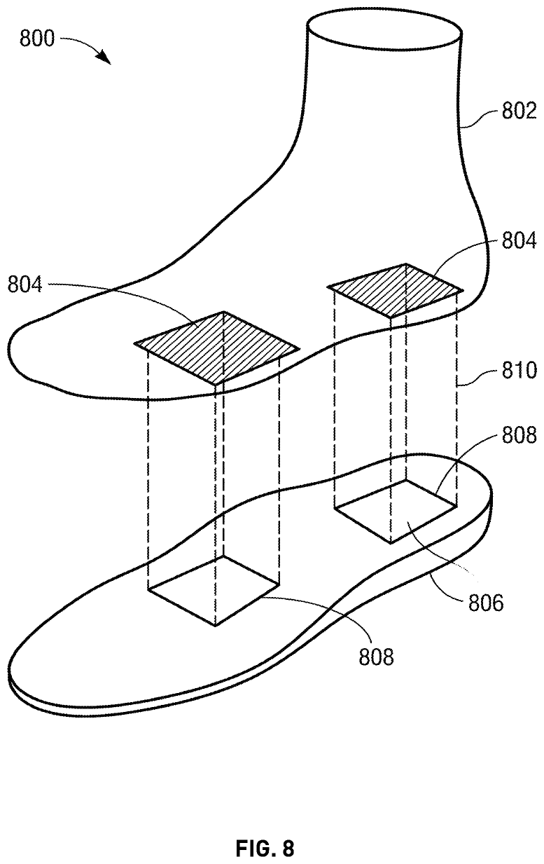

### FIG. 8: Foot Covering Registration System

FIG. 8 is a perspective view showing a registration system of a conductive foot covering and a smart insole, according to some examples.

FIG. 8 illustrates how electrical stimulation is effectively coupled to the user's foot. A smart insole 806, described above with reference to FIG. 7, serves as the primary structural and electronic foundation for delivering neurostimulation. The smart insole 806 utilizes insole stimulation pads 808, also described previously, which are embedded at specific target regions, such as the arch and heel, to deliver biphasic electrical waveforms intended to alleviate foot pain.

A conductive foot covering registration system 800 comprises a conductive foot covering 802 configured to interface with the smart insole 806. The conductive foot covering 802 includes one or more conductive textile patches 804 disposed on a plantar surface interface. In the depicted example, the conductive foot covering 802 is shaped as a wearable sock, with the conductive textile patches 804 positioned on the bottom surface corresponding precisely to the arch and heel regions of a human foot.

To optimize impedance reduction, the conductive textile patches 804 utilize specialized material compositions. For instance, in some embodiments, the conductive textile patches 804 are woven or knitted using silver-carbon fibers. These silver-carbon fibers provide a highly conductive, low-impedance pathway from the exterior of the conductive foot covering 802 directly to the user's skin. Furthermore, the fabric design of the conductive textile patches 804 is specifically engineered for moisture retention properties. By holding natural or induced sweat, the fabric provides a sustained, fluidic medium for delivery of electrical stimulation between the skin and the insole stimulation pads 808. In some examples, the smart insole 806 may include a low-power heater configured to gently warm the foot, intentionally inducing a micro-layer of sweat to further enhance conductivity and lower skin impedance at the plantar surface interface.

The conductive foot covering registration system 800 is configured such that a registration alignment 810 is established between the conductive textile patches 804 and the insole stimulation pads 808 when the user wears the conductive foot covering 802 and places their foot onto the smart insole 806. As shown by the vertical dashed lines in FIG. 8, the registration alignment 810 represents an electrical coupling interface. The conductive textile patches 804 are sized, shaped, and spatially distributed to match the underlying insole stimulation pads 808 perfectly. When the foot is placed on the smart insole 806, this spatial matching establishes a direct physical and electrical connection, ensuring that the stimulation waveforms are transmitted efficiently without requiring the user to make precise manual adjustments.

In various implementations, the conductive foot covering 802 may take alternative forms, such as a localized sleeve, an ankle brace, or a partial foot wrap, rather than a full sock. The conductive textile patches 804 may be formed by weaving or knitting conductive yarns, such as silver-coated nylon or stainless steel filament yarns, into the fabric structure of the conductive foot covering 802. The conductive textile patches 804 may alternatively utilize copper-infused threads, stainless steel micro-mesh, or conductive polymer coatings.

Additionally, the registration alignment 810 may be assisted by tactile feedback structures, magnetic alignment elements woven into the patches and embedded in the insole, or geometric contouring of the smart insole 806 that naturally guides the conductive foot covering 802 into the optimal electrical coupling position. Those skilled in the art will recognize that modifications and variations are possible without departing from the scope of the present disclosure.

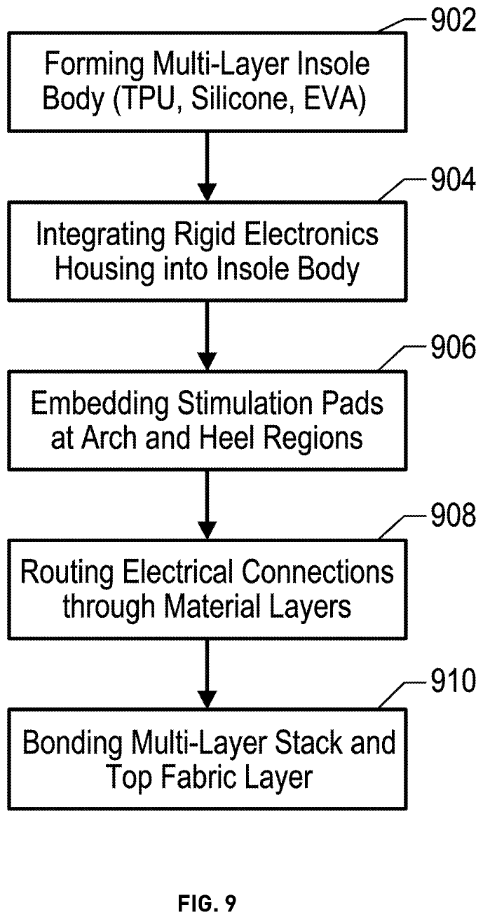

### FIG. 9: Insole Manufacturing Method

FIG. 9 is a flowchart showing a vertical sequence of a method 900 for manufacturing a smart insole, according to some examples.

The method 900 may include the set of assembly operations required to produce a durable, multi-layered neurostimulation insole. In some examples, method 900 is performed by an automated manufacturing system comprising molding apparatuses, pick-and-place robotics, and thermal bonding equipment.

The method 900 begins at operation 902, which forms a multi-layer insole body using materials such as thermoplastic polyurethane (TPU), silicone, and ethylene-vinyl acetate (EVA) foam. In some examples, an automated molding apparatus forms a base layer of TPU to provide structural rigidity, followed by a shock-absorbing layer of EVA foam or Poron. An insole body 702, described above with reference to FIG. 7, serves as the primary structural foundation that receives the subsequent electronic components. In at least one embodiment, a low-power heating element is embedded between the molded silicone and foam layers during this operation. This heating element is configured to induce localized sweat on the plantar surface of the user's foot, thereby increasing skin moisture and improving electrical conductivity for the neurostimulation process. Some embodiments may utilize different assembly sequences for embedding the heating element, such as integrating it directly within a top fabric layer or utilizing a flexible printed heater circuit.

Method 900 proceeds to operation 904, which integrates a rigid electronic housing into the insole body. During this operation, an assembly apparatus inserts an electronics housing 714, described above with reference to FIG. 7, into a corresponding cavity molded within the insole body 702. The structural integration of the rigid housing is designed to withstand high compressive loads generated during ambulatory states, such as walking or running. In some implementations, the electronics housing 714 is formed from rigid polyethylene terephthalate glycol (PETG) or polylactic acid (PLA) and is secured within the cavity using industrial adhesives, snap-fit retention features, or ultrasonic welding.

Method 900 may continue to operation 906 of embedding stimulation pads at arch and heel regions. A robotic placement system positions stimulation pads 518, described above with reference to FIG. 5, into designated recesses corresponding to the arch and heel regions of the foot to ensure strategic pad placement. To maintain long-term durability and conductivity, an electrode-coating application method is used during or prior to this operation. Specifically, a highly conductive coating is applied to the stimulation pads 518 and subjected to a specialized curing and baking process. This thermal curing ensures the conductive coating does not wear down or delaminate under the repeated cyclic loading and shear forces exerted by the user's foot. In alternative implementations, the stimulation pads 518 may comprise conductive textiles or hydrogel-infused carbon matrices that are mechanically stitched rather than chemically bonded.

Method 900 proceeds to operation 908, which routes electrical connections through the material layers. An automated routing tool embeds flexible printed circuits or conductive wires to establish electrical communication between the electronics housing 714 and the stimulation pads 518. In some implementations, the conductive wires comprise stranded silicone hookup wire configured to provide heat resistance sufficient for soldered interconnections to the flexible printed circuit board and sufficient flexibility to be routed through small cavities within the material layers. The protection of these embedded connections is critical to prevent fatigue failures; thus, the conductive traces are typically routed in a serpentine pattern and sandwiched between a flexible silicone support layer and an EVA foam cushioning layer to isolate them from mechanical strain.

Method 900 concludes with operation 910, which bonds the multi-layer stack and a top fabric layer. Thermal bonding equipment applies controlled heat and pressure to seal the internal components, laminating a breathable, moisture-wicking top fabric layer over the assembly to provide a seamless and comfortable user interface. In some implementations, adhesive-based bonding is used in place of or in combination with thermal bonding to join the layers of the multi-layer stack. For example, a silicone primer may be applied to a thermoplastic polyurethane (TPU) base layer and a silicone adhesive may be applied to an adjacent silicone support layer to bond the TPU base layer to the silicone support layer. A silicone adhesive may further be applied between the silicone support layer and an overlying fabric layer to bond the silicone support layer to the fabric layer, and a silicone adhesive may further be applied to bond conductive patches to the fabric layer. An adhesive selected for compatibility with open-cell or closed-cell polymer foam, such as a polyurethane-based or contact-type foam adhesive, may be applied to bond a fabric layer to an adjacent foam cushioning layer. Those skilled in the art will recognize that modifications and variations are possible without departing from the scope of the present disclosure.

Although the example method depicts a particular sequence of operations, the sequence may be altered without departing from the scope of the present disclosure. For example, some operations may be performed in parallel or in a different sequence that does not materially affect method 900. In some examples, different components of an example device or system that implements method 900 may perform functions at substantially the same time or in a specific sequence.

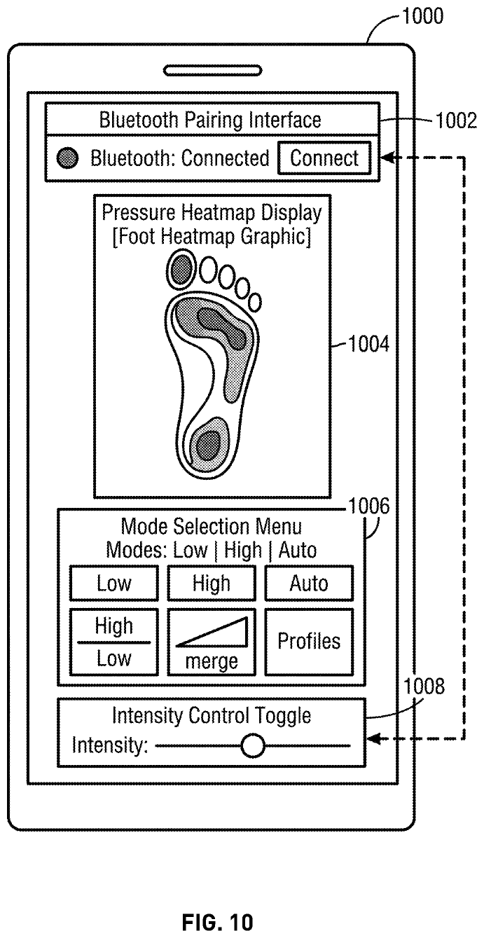

### FIG. 10: Mobile Application User Interface

FIG. 10 is a context diagram showing a top-level view of a mobile application user interface 1000 within a smartphone environment, according to some examples.

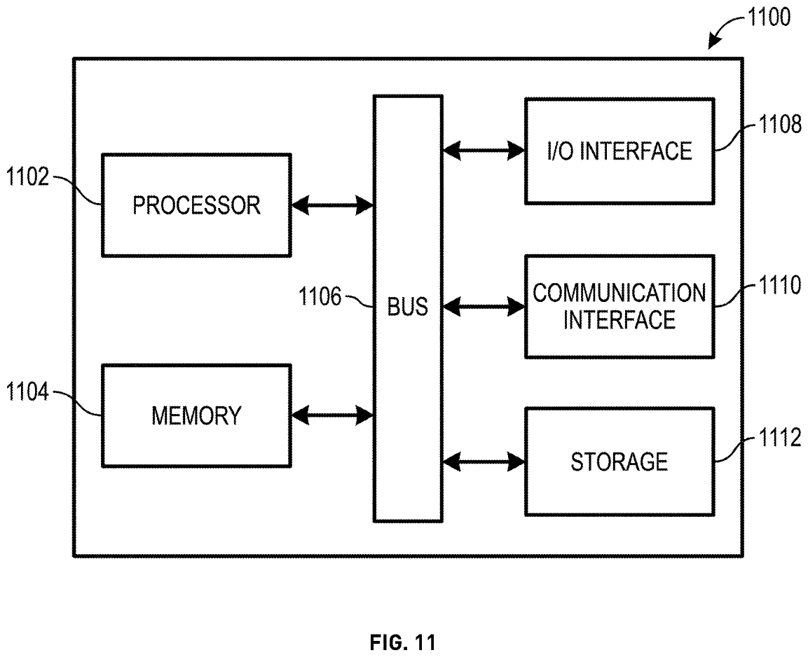

Users may interact with the neurostimulation system via a digital interface. The mobile application user interface 1000 may be presented on a computing device 1100, described below with reference to FIG. 11. The processing capabilities of the computing device 1100 are utilized to execute the application logic, render the graphical user interface, and process incoming telemetry streams into actionable visual feedback. By leveraging local computing resources, the mobile application maintains low-latency responsiveness when adjusting stimulation settings based on user input or automated algorithms.

At the top of the screen within the user interface 1000 a Bluetooth pairing interface 1002 is depicted as a horizontal bar containing a connection status indicator and a ‘Connect’ button. The Bluetooth pairing interface 1002 establishes a logical connection with the wireless communication module 504, described above with reference to FIG. 5. This wireless synchronization used critical for bidirectional data transfer, enabling the mobile application to send stimulation parameters to the insole hardware while simultaneously receiving real-time sensor telemetry. Without this continuous communication link, the dynamic adjustments and live feedback presented in the user interface would not be possible. In some implementations, the Bluetooth pairing interface 1002 establishes concurrent logical connections with the wireless communication module 504 of each of a pair of smart insoles 102 worn on respective feet of the user, and the mobile application synchronizes stimulation intensity settings across both smart insoles 102 such that adjustments made via the intensity control toggle 1008 are applied in parallel to each insole to provide a balanced bilateral stimulation experience. In some implementations, the wireless communication module 504 comprises or is coupled to a Bluetooth range extender configured to maintain the logical connection with the mobile application at a separation distance of up to approximately 10 meters (approximately 32 feet).

Centered in the upper half of the user interface 1000 is a pressure heatmap display 1004, which provides visual feedback of plantar pressure distribution. The pressure heatmap display 1004 consists of a stylized outline of a human foot with shaded regions representing different pressure intensities detected by the insole sensors. The pressure heatmap display 1004 visually represents data captured by the sensor matrix 710, described above with reference to FIG. 7. As the user walks or stands, the sensor matrix 710 continuously streams localized pressure values to the mobile application, which translates these discrete data points into a continuous, color-coded visual gradient. This allows the user to immediately perceive their weight-bearing regions and visually verify that the insole is accurately detecting varying degrees of foot contact during different phases of gait.

Directly below the pressure heatmap display 1004, a mode selection menu 1006 is presented as a grid of buttons labeled with various stimulation profiles, such as ‘Low’, ‘High’, and ‘Auto’. Spanning the width of the lower section of the screen is an intensity control toggle 1008, illustrated as a horizontal slider bar with a circular handle for adjusting the magnitude of the neurostimulation signal. A logical control link flows from the Bluetooth pairing interface 1002 to the intensity control toggle 1008, establishing that the connection status dictates the control availability within the interface. In some examples, user control of stimulation intensity can be automated; selecting the ‘Auto’ profile in the mode selection menu 1006 causes the application to dynamically scale the output of the intensity control toggle 1008 in direct response to the real-time pressure fluctuations shown in the pressure heatmap display 1004.

In some implementations, the mobile application user interface 1000 further includes a device status panel displayed within the smartphone environment. The device status panel may be positioned adjacent to the Bluetooth pairing interface 1002 or as a collapsible overlay accessible from a status bar icon. The device status panel is configured to receive telemetry data from the smart insole 102 via the wireless communication module 504 and to render the received telemetry as human-readable status indicators. For example, the device status panel may display a battery level indicator, such as a graphical battery icon with a fill level proportional to the remaining charge of the batteries 410, or a numerical percentage representing the remaining charge. The device status panel may further display a power state indicator showing whether the smart insole 102 is currently powered on or powered off.

In some examples, the power state indicator is rendered as a colored icon or text label (e.g., “Active” or “Standby”) that updates in response to state change notifications received from the microcontroller 502. The device status panel may additionally display a stimulation active indicator showing whether the stimulation circuit is currently delivering electrical pulses through the stimulation pads 518, enabling the user to confirm that therapy is in progress without relying solely on physical sensation. In some implementations, the device status panel displays an electrode contact quality indicator derived from impedance measurements taken by the microcontroller 502 at the electrode-skin interface. The electrode contact quality indicator may be rendered as a qualitative scale (e.g., “Good,” “Fair,” “Poor”) or as a numerical impedance value in ohms, providing the user with feedback on whether the conductive foot covering 802 and the stimulation pads are properly aligned and in adequate contact with the skin. In further implementations, the device status panel may display a session timer showing the elapsed duration of the current stimulation session and a cumulative usage counter showing the total stimulation time logged over a user-configurable reporting period, such as a day, a week, or a month. The mobile application 106 may also generate push notifications or audible alerts on the user device 104 when the battery level of the smart insole 102 falls below a configurable threshold, when the wireless connection between the user device 104 and the smart insole 102 is lost, or when the stimulation circuit detects a fault condition such as an open electrode circuit or an overcurrent event.

### FIG. 11: Computing Device Architecture

FIG. 11 is a block diagram showing a top-level view of a generic computing device 1100, according to some examples.

The computing device 1100 can serve as the technical substrate for various system components, such as a user device 104 and a cloud server 110, described above with reference to previous figures. The user device 104 relies on the computing device 1100 architecture to execute a mobile application, process user inputs, and maintain wireless connections with a smart insole. Similarly, the cloud server 110 utilizes the computing device 1100 architecture to aggregate stimulation data, perform remote analytics, and distribute firmware updates across a network. Within the computing device 1100, a bus 1106 acts as the primary communication backbone, providing bidirectional data flow and interconnectivity between all internal components.

A processor 1102 is communicatively coupled to the bus 1106 and is configured to execute control logic and processing instructions. In some examples, the processor 1102 comprises a central processing unit (CPU), a microprocessor, or an application-specific integrated circuit (ASIC) that handles the core computational tasks of the system. A memory 1104, also coupled to the bus 1106, provides temporary and working storage for the processor 1102. The memory 1104 may store active user thresholds, real-time intensity settings, and transient sensor data received during a neurostimulation session. The memory 1104 typically includes volatile storage mediums such as random-access memory (RAM) or dynamic RAM (DRAM), enabling the rapid read and write operations required for real-time therapy control.

A storage 1112 provides non-volatile data retention for the computing device 1100.

The storage 1112 may include solid-state drives (SSDs), flash memory, or magnetic disk drives configured to permanently house operating systems, mobile application binaries, and historical user foot heatmap data. An I/O interface 1108 facilitates interaction between the computing device 1100 and external input/output mechanisms. For example, the I/O interface 1108 manages signals from a user interface 522, described above with reference to previous figures. The user interface 522 depends on the I/O interface 1108 to translate physical button presses or touch-screen interactions into digital commands that the processor 1102 can interpret to adjust stimulation modes or intensities.

A communication interface 1110 handles external network and device connectivity. In some examples, the communication interface 1110 comprises wireless transceivers configured to manage Bluetooth Low Energy (BLE) connections, Wi-Fi signals, or cellular data links. This allows the computing device 1100 to transmit stimulation parameters locally to the smart insole or sync historical usage data remotely with cloud databases. Those skilled in the art will recognize that modifications and variations are possible without departing from the scope of the present disclosure, and that the computing device 1100 may include additional or fewer components depending on its specific implementation as a mobile device, a server, or an embedded controller.

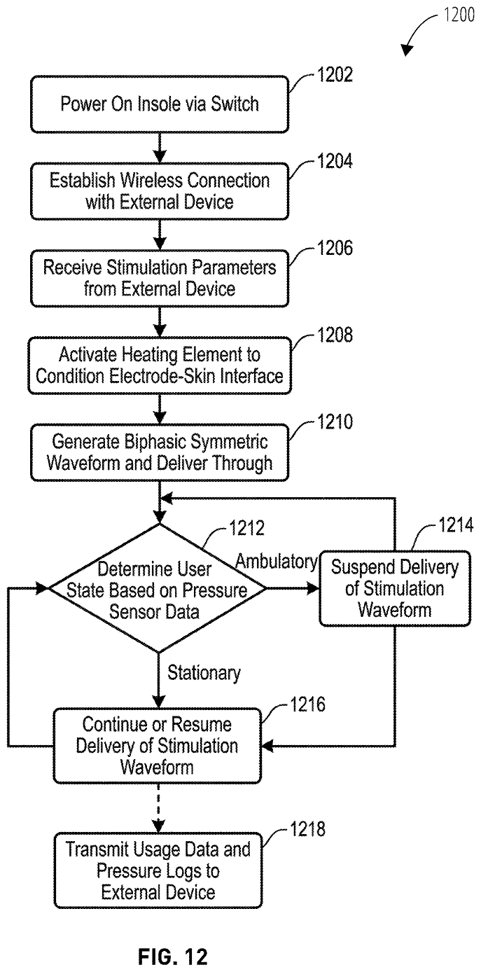

### FIG. 12: Method of Operating the Therapeutic Insole

FIG. 12 is a flowchart showing a method 1200 for operating the therapeutic insole during a therapy session, according to some examples.

Method 1200 describes the runtime operational sequence performed by the therapeutic insole from initial power-on through active therapy delivery, including a continuous pressure-sensor-based monitoring loop that governs when stimulation is active. In some examples, method 1200 is performed by the nerve stimulation circuit 500, described above with reference to FIG. 5, executing on the microcontroller 502 in coordination with the mobile application 106, described above with reference to FIGS. 1 and 10.

Method 1200 begins at operation 1202, in which a user powers on the therapeutic insole. The user activates the insole via the switch 414, described above with reference to FIG. 4. Upon activation, the microcontroller 502 initializes its internal registers, loads stored configuration parameters from non-volatile memory, and performs a self-diagnostic check of connected peripherals, including the pulse generator 510, the sensor interface 514, and the wireless communication module 504. Upon completion of initialization, the microcontroller 502 drives the LED indicator 520 to emit a steady illumination in a first color (e.g., green) to indicate that the device is powered on and operating. In some embodiments, the microcontroller 502 also drives the LED indicator 520 to convey battery charge status. For example, the LED indicator 520 may emit a steady illumination in a second color (e.g., blue) when the battery charge level is above a predetermined threshold, or may emit a blinking pattern in a third color (e.g., red) when the battery charge level falls below the predetermined threshold.

Method 1200 proceeds to operation 1204, in which the wireless communication module 504 establishes a wireless connection with an external device. Upon power-on, the wireless communication module 504 enters a pairing mode in which it broadcasts advertising packets over a short-range wireless protocol, such as Bluetooth Low Energy (BLE). During pairing mode, the LED indicator 520 may emit a rapid blinking pattern to indicate that the wireless communication module 504 is awaiting a connection request from the user device 104. The mobile application 106, executing on the user device 104, detects the advertising packets and initiates a connection handshake with the wireless communication module 504. Upon establishment of a bidirectional communication link, the LED indicator 520 transitions to a steady illumination or a slow periodic blink to indicate that a wireless connection has been established. The bidirectional link enables the mobile application 106 to transmit stimulation control signals to the microcontroller 502 and simultaneously enables the microcontroller 502 to transmit telemetry data, including battery status, sensor readings, and stimulation logs, to the mobile application 106 for display on the user device 104. In some implementations, if the wireless communication module 504 does not receive a connection request within a predetermined timeout period, the microcontroller 502 transitions the wireless communication module 504 from pairing mode to a low-power standby mode to conserve battery power, and the insole operates using previously stored stimulation parameters or default parameters without requiring an active wireless connection.

Method 1200 proceeds to operation 1206, in which the microcontroller 502 receives stimulation parameters from the external device. The mobile application 106 transmits stimulation control signals to the wireless communication module 504, which relays the signals to the microcontroller 502. The stimulation control signals may specify at least one of a stimulation intensity level, a stimulation pulse frequency, a stimulation mode (e.g., Low, High, or Auto), a pulse width, or a stimulation on/off state. The microcontroller 502 receives these parameters and stores them in working memory for use in subsequent waveform generation. In some embodiments, the stimulation control signals further specify a target current setpoint for use in a current-controlled stimulation mode, as described above with reference to FIG. 5. In some implementations, the mobile application 106 transmits updated stimulation control signals during an active therapy session, and the microcontroller 502 applies the updated parameters to the waveform generation in real time without interrupting the ongoing stimulation delivery. In embodiments where the wireless connection is not established or is lost during operation, the microcontroller 502 proceeds using the most recently stored parameters or factory-default parameters.

Method 1200 proceeds to operation 1208, which is an electrode conditioning operation performed in embodiments that include a heating element, as described above with reference to FIGS. 3 and 5. In such embodiments, the microcontroller 502 activates the heating element for a predetermined conditioning period prior to activating the stimulation circuit. The heating element applies localized thermal energy to the plantar surface of the user's foot through the surface layer of the multi-layer body, inducing a controlled amount of perspiration at the electrode-skin interface. During the conditioning period, the microcontroller 502 monitors skin impedance at the electrode-skin interface by measuring the ratio of an applied test voltage to the resulting current through the stimulation pads 518.

When the monitored skin impedance falls below a threshold value stored in the microcontroller 502, indicating that sufficient moisture is present at the electrode-skin interface for transcutaneous delivery of electrical stimulation, the microcontroller 502 deactivates the heating element and proceeds to operation 1210. In some implementations, the microcontroller 502 regulates the temperature of the heating element within a range of approximately 37 degrees Celsius to 40 degrees Celsius during the conditioning period to induce localized perspiration without causing thermal discomfort to the user. In embodiments that do not include a heating element, method 1200 proceeds directly from operation 1206 to operation 1210.

Method 1200 proceeds to operation 1210, in which the microcontroller 502 generates a biphasic symmetric stimulation waveform and delivers the waveform through the stimulation electrodes. The microcontroller 502 generates a pulse-width-modulated (PWM) signal based on the stimulation parameters received in operation 1206. The PWM signal is provided to the pulse generator 510, where the operational amplifier 512 inverts and offsets the unipolar PWM signal to produce a biphasic symmetric waveform having positive and negative phases of substantially equal amplitude relative to a zero-baseline reference, as described above with reference to FIG. 6. The biphasic symmetric waveform is delivered through the stimulation pads 518 to the plantar surface of the user's foot via the arch stimulation pad 208 and the heel stimulation pad 210, described above with reference to FIG. 2. The biphasic symmetry ensures that the net electrical charge delivered through the stimulation electrodes over each complete cycle of the waveform is substantially zero, preventing accumulation of ions at the electrode-skin interface and reducing the risk of skin irritation or electrochemical tissue damage during prolonged therapy sessions. In embodiments operating in the current-controlled mode, the microcontroller 502 concurrently monitors the voltage across a current-sensing element in the output path and adjusts the output voltage of the pulse generator 510 to maintain the target current setpoint despite fluctuations in skin impedance.

Concurrently with the stimulation delivery of operation 1210, method 1200 proceeds to decision 1212, in which the microcontroller 502 determines whether the user's foot is in a stationary state or an ambulatory state based on pressure sensor data. The microcontroller 502 continuously receives pressure data from the sensor matrix 710, described above with reference to FIG. 7, via the sensor interface 514. The sensor interface 514 samples the pressure sensors 516 at a rate of 50 Hz to 200 Hz and applies digital low-pass filtering to smooth the pressure data before state evaluation. The microcontroller 502 evaluates the filtered pressure data against stored threshold values to classify the user's movement state. The determination may be based on one or more of: (a) an absolute pressure magnitude at one or more sensor locations exceeding or falling below a predefined threshold measured in millipascals (mPa), (b) a rate of change of the pressure signal over time that is indicative of gait cycles, (c) a periodic pressure pattern consistent with a walking cadence, or (d) a differential pressure reading between two or more sensor locations, such as between the heel pressure sensor 704 and the forefoot pressure sensor 708, that indicates sequential weight transfer characteristic of ambulation. To prevent rapid, unintended stimulation cycling during the dynamic pressure fluctuations of a standard gait cycle, the microcontroller 502 implements debounce logic that requires the detected state to persist for a minimum duration before triggering a state transition.

If the microcontroller 502 determines at decision 1212 that the user's foot is in the ambulatory state, method 1200 proceeds to operation 1214, in which the microcontroller 502 suspends delivery of the biphasic symmetric stimulation waveform. The microcontroller 502 deactivates the pulse generator 510, and the stimulation pads 518 cease receiving current. The suspension of stimulation during the ambulatory state serves to maintain safety and consistent electrode contact. During ambulation, the foot undergoes repeated cycles of heel strike, midstance, and toe-off, causing the foot to shift, flex, and partially lift relative to the insole surface. These movements disrupt the contact interface between the stimulation pads and the skin, which can result in inconsistent delivery of electrical stimulation or localized current concentration at partial-contact points. By suspending stimulation during the ambulatory state, the system avoids delivering current through a misaligned or partially contacted electrode-skin interface. Following suspension at operation 1214, the microcontroller 502 continues to monitor pressure data from the sensor matrix 710 and returns to decision 1212 to re-evaluate the user's movement state.

If the microcontroller 502 determines at decision 1212 that the user's foot is in the stationary state, or if the microcontroller 502 detects at decision 1212 that the user's foot has returned to the stationary state following a prior ambulatory suspension, method 1200 proceeds to operation 1216, in which the microcontroller 502 continues or resumes delivery of the biphasic symmetric stimulation waveform through the stimulation pads 518. In the stationary state, the stimulation pads maintain consistent contact alignment with the targeted nerve regions on the plantar surface, allowing for reliable and uniform delivery of electrical stimulation through the skin interface. The microcontroller 502 resumes waveform generation using the same stimulation parameters that were active prior to the ambulatory suspension, or using updated parameters if new stimulation control signals were received from the mobile application 106 during the suspension period. Following resumption at operation 1216, the microcontroller 502 returns to decision 1212 to maintain continuous monitoring of the user's movement state. The loop between decision 1212, operation 1214, and operation 1216 operates continuously throughout the therapy session, such that the stimulation delivery automatically adapts to transitions between stationary and ambulatory states without requiring manual intervention by the user.

In some embodiments, the microcontroller 502 uses pressure data from the sensor matrix 710 to adjust stimulation parameters during the stationary state, in addition to the binary stationary/ambulatory determination described above. For example, while the user is standing, the microcontroller 502 may detect that the pressure profile indicates a concentration of weight on the medial arch region and, in response, increase the stimulation intensity or pulse frequency delivered through the arch stimulation pad 208. Conversely, if the pressure profile indicates a shift of weight toward the heel, the microcontroller 502 may increase stimulation through the heel stimulation pad 210. This pressure-responsive modulation allows the stimulation circuit to adapt the therapeutic output to the user's real-time weight distribution.

During the therapy session, method 1200 includes operation 1218, in which the microcontroller 502 logs session data and transmits the logged data to the external device. The microcontroller 502 records time-stamped entries of detected stationary and ambulatory state transitions, cumulative time spent in each state, cumulative stimulation delivery time, the pressure distribution profile during stationary periods, battery charge level readings, and any fault conditions detected during the session, such as open electrode circuits, overcurrent events, or loss of wireless connection. The microcontroller 502 periodically transmits the logged data to the mobile application 106 via the wireless communication module 504. In some implementations, the mobile application 106 processes and displays the session data to provide the user with feedback regarding the total time spent in stimulation-eligible postures during a given day or session, the total stimulation time delivered, and the battery charge remaining. The mobile application 106 may further route the session data through the wireless network 108 to the cloud server 110, described above with reference to FIG. 1, for aggregation, longitudinal analysis, and remote monitoring by a healthcare provider. In some embodiments, the mobile application 106 generates push notifications or audible alerts on the user device 104 when the battery level of the therapeutic insole falls below a configurable threshold, when the wireless connection between the user device 104 and the therapeutic insole is lost, or when the stimulation circuit detects a fault condition.

Although the example method depicts a particular sequence of operations, the sequence may be altered without departing from the scope of the present disclosure. For example, operation 1204 (establishing a wireless connection) may be performed before, after, or concurrently with operation 1208 (activating the heating element). In some implementations, the insole may begin delivering stimulation at operation 1210 using stored default parameters without first performing operation 1206, and may subsequently receive updated parameters from the mobile application during an active session. In some examples, operation 1218 (data logging and transmission) may be performed continuously throughout the session rather than as a discrete final step. Furthermore, in embodiments that do not include pressure sensors, the decision 1212 and operations 1214 and 1216 may be omitted, and the insole may deliver stimulation continuously until the user manually deactivates the device via the switch 414 or via a command from the mobile application 106. Those skilled in the art will recognize that modifications and variations are possible without departing from the scope of the present disclosure.

### Technical Considerations

The present disclosure addresses technical challenges associated with traditional orthotic devices, which often rely on passive mechanical support that may fail to provide immediate pain relief for conditions such as flat feet (pes planus). In some embodiments, the disclosed technology provides an active therapeutic solution by integrating electrical nerve stimulation directly into a supportive, multi-layer insole structure. By utilizing a multi-layer body comprising specific density gradients-such as a support layer for structural integrity and a cushioning layer for shock absorption-the system can provide both mechanical correction and electro-chemical pain signal modulation. This dual-action approach is configured to block pain signals and stimulate the release of natural endorphins, offering a more comprehensive treatment than static arch supports.

Various design choices and implementations may be employed to optimize therapeutic effect and user comfort in accordance with the present disclosure. For example, the base structure may be constructed from flexible polymer materials such as polyethylene terephthalate glycol (PETG) or thermoplastic polyurethane (TPU) to balance structural rigidity with the necessary flexibility for natural gait cycles. In other examples, the base structure or an underlying shell may be constructed from materials used in custom orthotic fabrication, including polyethylene (for example, Orthelon or Suborthelon), polypropylene, graphite or other carbon-fiber composites embedded in an acrylic matrix, acrylic copolymer shell materials (for example, Polydur), or cork-based composites (for example, cork combined with thermoplastic polyolefin, cork combined with ethylene-vinyl acetate, or rubberized cork). In some examples, the cushioning layer may incorporate materials such as Poron or ethylene-vinyl acetate (EVA) foam to maintain durability and energy return under repetitive loading. In further examples, the cushioning layer or an arch-fill layer disposed beneath the support layer may incorporate cork-based composites or polyurethane open-cell foam (for example, Poron) to tune the rigidity of the assembly while providing shock absorption. In still further examples, a surface layer or top cover overlying the cushioning layer may comprise closed-cell polyethylene foam (for example, Plastazote), EVA of selected durometer, polyurethane-based soft-tissue replacement foam (for example, PPT or Poron), neoprene-based shock-absorbing foam (for example, Spenco), vinyl (for example, Naugahyde), or leather or suede, selected to provide a desired combination of moldability, shock absorption, durability, and moisture management at the foot-device interface. Furthermore, the stimulation circuit may be configured to generate a biphasic waveform, which can reduce the risk of skin irritation or electrolytic burns compared to monophasic pulses. In some embodiments, the stimulation circuit may alternatively operate in a current-controlled mode rather than a voltage-controlled mode, in which a feedback loop monitors the delivered current via a sensing element in the output path and adjusts the driving voltage to maintain a target current setpoint, thereby providing consistent stimulation delivery despite fluctuations in skin impedance caused by variations in perspiration, skin dryness, electrode contact area, or changes in contact pressure during use. In some implementations, the insole may further include a low-power heating element configured to induce a localized sweat response, thereby increasing the electrical conductivity between the stimulation electrodes and the user's skin for more efficient signal delivery.

Performance considerations and trade-offs are managed through integrated sensing and control logic in at least one embodiment. The system may include one or more pressure sensors configured to detect transitions between stationary states (e.g., sitting or standing) and ambulatory states (e.g., walking or running). This allows the stimulation circuit to pause or modulate delivery when the user is in motion, preventing potential interference with motor control or gait stability while prioritizing treatment during sedentary periods. Additionally, a dual-battery configuration may be utilized in some implementations to provide a balance between device longevity and the slim profile required for footwear integration. Wire routing channels formed within the multi-layer body ensure that electrical interconnections remain protected from mechanical stress, friction, and moisture, enhancing the overall reliability and mean time between failures (MTBF) of the system across diverse environmental conditions.

The disclosed technology is designed for scalability and extensibility across various footwear platforms and therapeutic applications. While described in some examples in the context of flat foot pain, the stimulation parameters-such as pulse frequency, pulse width, and intensity-may be adjusted via an external device and wireless communication module to treat other neuropathic or musculoskeletal conditions. In some examples, the modular nature of the electronics enclosure and electrode placement allows the system to be adapted for different shoe sizes or specialized athletic footwear. The software application interface may also support real-time telemetry and data logging, enabling users or healthcare providers to monitor usage patterns and optimize stimulation protocols for individualized care. These configurations ensure the system remains adaptable to evolving clinical requirements and user preferences.

### Use Cases

The following use cases describe various scenarios in which the disclosed system and method may be deployed. These examples are illustrative and do not limit the scope of the present disclosure.
Chronic Pain Management for Pes Planus: In some embodiments, the disclosed system is utilized by individuals suffering from flat feet (pes planus) to manage chronic pain localized in the arch and ankle regions. As a user engages in daily activities, the base structure and multi-layer body provide mechanical support to the natural arch of the foot, reducing pressure on the ankle bone. When the user experiences a flare-up of sharp pain, the stimulation circuit delivers a biphasic electrical waveform through the stimulation electrodes. This electrical stimulation may function to block pain signals from reaching the brain and stimulate the release of natural endorphins. Because the system utilizes a biphasic waveform, the user can receive prolonged or repeated stimulation sessions while minimizing the risk of skin irritation or burns.
Automated Stationary Stimulation Based on Pressure Sensing: In at least one embodiment, the therapeutic insole utilizes integrated pressure sensors to optimize the timing of electrical stimulation. The disclosed system may detect a change in force distribution across the plantar support surface that corresponds to a transition between an ambulatory state (e.g., walking) and a stationary state (e.g., sitting or standing). In some implementations, the software application on the external device is configured to trigger stimulation only when the pressure sensors indicate the user is substantially stationary. This ensures that the electrodes maintain consistent contact with the skin and that the muscle stimulation does not interfere with the user's gait or balance during movement.
Conductivity Enhancement in Dry Environments: In some examples, the disclosed system is deployed to assist users with low skin conductivity, which can occur due to dry skin or cold environments. The therapeutic insole may include a low-power heating element integrated within the multi-layer body. When the stimulation circuit detects high impedance between the stimulation electrodes, or upon a manual command from the external device, the heating element may be activated to induce a localized, slight amount of perspiration. This moisture serves as a natural conductive medium, lowering the impedance at the electrode-skin interface and allowing the electrical stimulation signals to penetrate the tissue more effectively and comfortably.
Post-Activity Recovery and Rehabilitation: In some implementations, the disclosed system is used as a recovery tool for athletes or patients undergoing physical therapy for foot-related injuries. Following a period of physical exertion, a user may insert the therapeutic insole into their footwear or a recovery boot. Through the software application on the external device, the user can select specific stimulation profiles, such as varying pulse frequencies or intensities, tailored to muscle recovery. The support layer and cushioning layer, which may include materials such as Poron or EVA foam, provide a comfortable interface for the foot while the stimulation circuit delivers pulses designed to alleviate muscle fatigue and prevent the onset of stiffness.
Customizable Orthotic Integration for Varied Footwear: According to some embodiments, the disclosed system provides a modular solution for users who require therapeutic stimulation across different types of footwear. Because the electronics enclosure is integrated into the heel portion of the base structure and the wire routing channels are protected within the multi-layer body, the insole maintains a low profile similar to a standard orthotic. This allows the user to transfer the therapeutic insole between athletic shoes, dress shoes, or work boots. The wireless communication module ensures that the user can control the stimulation parameters discreetly via a smartphone or wearable device, regardless of the footwear being worn, providing a seamless transition between different daily environments.

### GLOSSARY

As used herein, the term “therapeutic insole” may refer to a removable orthotic insert configured for placement within footwear that provides physiological or medical benefits to a user, for example through the delivery of controlled electrical stimulation to the plantar surface of the foot.
As used herein, the term “plantar support surface” may refer to the superior-facing surface of the insole body shaped, for example, to generally conform to the contours of the underside (plantar aspect) of a human foot, including the heel, arch, and forefoot regions.
As used herein, the term “stimulation circuit” may refer to an assembly of electronic components, including, for example, but not limited to, a microcontroller, power source, and pulse generator, cooperatively configured to generate, modulate, and deliver electrical current for the purpose of nerve or muscle stimulation.
As used herein, the term “flexural modulus” may refer to a physical property of a material representing its tendency to bend or its stiffness when a force is applied, specifically the ratio of stress to strain in flexural deformation.
As used herein, the term “pulse-width-modulated (PWM) signal” may refer to a technique used to encode a message or control power delivery by varying the duty cycle or width of electrical pulses within a signal stream.
As used herein, the term “net-zero charge” may refer to an electrical waveform characteristic where the total amount of positive electrical charge delivered during a stimulation phase is substantially equal to the total amount of negative charge delivered, thereby preventing the accumulation of residual charge in biological tissue.
As used herein, the term “conductive fabric patches” may refer to textile materials integrated with conductive elements-such as silver, stainless steel, or carbon fibers-configured to provide an interface for transferring electrical current from the internal electrodes of the insole to the user's skin or a conductive foot covering.
As used herein, the term “trim-to-fit zone” may refer to an anterior region of the insole, typically located at the toe portion, constructed from materials devoid of electronic components or wiring, thereby allowing a user to manually cut the material to adjust the length of the insole to fit specific footwear.
As used herein, the term “operational amplifier” may refer to a high-gain electronic voltage amplifier with a differential input and, usually, a single-ended output, used within the stimulation circuit to amplify or condition control signals into therapeutic electrical pulses.
As used herein, the term “anatomical zones” may refer to specific regions of the human foot defined by underlying skeletal or neural structures, such as the calcaneus (heel), the medial longitudinal arch, and the metatarsal (forefoot) regions.

### Clauses

In view of the above-described implementations of subject matter this application discloses the following list of examples, wherein one feature of an example in isolation or more than one feature of an example, taken in combination and, optionally, in combination with one or more features of one or more further examples are further examples also falling within the disclosure of this application.
Example 1 is a therapeutic insole configured to be inserted into footwear, the therapeutic insole comprising a base structure defining a plantar support surface shaped to conform to an underside of a human foot, a multi-layer body disposed on the base structure, the multi-layer body comprising a support layer, a cushioning layer disposed on the support layer, and a surface layer disposed on the cushioning layer, at least two stimulation electrodes embedded within the multi-layer body, a first electrode positioned at a location corresponding to an arch region of the foot and a second electrode positioned at a location corresponding to a heel region of the foot, wherein the at least two stimulation electrodes are accessible at or through the surface layer, an electronics enclosure integrated into the base structure at a heel portion thereof, the electronics enclosure housing a stimulation circuit comprising a microcontroller and one or more power sources, and one or more wire routing channels formed within the multi-layer body and extending from the electronics enclosure to each of the at least two stimulation electrodes, wherein the stimulation circuit is configured to deliver electrical stimulation through the at least two stimulation electrodes.
Example 2 according to example 1, wherein the stimulation circuit further comprises an operational amplifier configured to receive a pulse-width-modulated signal from the microcontroller and to invert and offset the pulse-width-modulated signal to produce a biphasic symmetric stimulation waveform delivered through the at least two stimulation electrodes such that electrical current flows in alternating polarity between the first electrode and the second electrode.
Example 3 according to any one or more of examples 1 to 2, wherein the electronics enclosure comprises an enclosure body and a lid secured over the enclosure body, the lid formed from a material having a flexural modulus sufficient to resist deformation into contact with components of the stimulation circuit when the insole is subjected to a distributed compressive load corresponding to a body weight of a user, and wherein the enclosure body includes one or more internal support structures positioned between the lid and the stimulation circuit to distribute compressive loads away from the stimulation circuit components.
Example 4 according to any one or more of examples 1 to 3, wherein the support layer comprises a molded elastomeric material, the cushioning layer comprises at least one of ethylene vinyl acetate foam or an open-cell polyurethane foam, and the base structure comprises a thermoplastic polyurethane.
Example 5 according to any one or more of examples 1 to 4, wherein the one or more wire routing channels are enclosed channels formed within the support layer and extend longitudinally from the electronics enclosure toward the arch region, routing separate conductive pathways to each of the at least two stimulation electrodes.
Example 6 according to any one or more of examples 1 to 5, further comprising conductive fabric patches disposed on the surface layer at positions aligned with the at least two stimulation electrodes, the conductive fabric patches formed from a moisture-retaining conductive material configured to provide an electrical conduction path between the at least two stimulation electrodes and a plantar surface of a user's foot and to retain moisture at the electrode-skin interface to sustain delivery of electrical stimulation under low-perspiration conditions.
Example 7 according to any one or more of examples 1 to 6, wherein the stimulation circuit further comprises a heating element positioned adjacent to at least one of the at least two stimulation electrodes, the heating element configured to apply localized thermal energy to induce perspiration at an electrode-skin interface, thereby reducing skin impedance and improving transcutaneous pulsed delivery of electrical stimulation through the at least two stimulation electrodes.
Example 8 according to example 7, wherein the heating element is a resistive heating element embedded within the multi-layer body between the surface layer and the cushioning layer at a position corresponding to the at least one of the at least two stimulation electrodes, and wherein the microcontroller is configured to regulate a temperature of the heating element within a range of approximately 37 degrees Celsius to 40 degrees Celsius to induce localized perspiration without causing thermal discomfort to a user.
Example 9 according to any one or more of examples 7 to 8, wherein the microcontroller is configured to activate the heating element for a predetermined conditioning period prior to activating the stimulation circuit, the conditioning period being of a duration sufficient to establish a moisture layer at the electrode-skin interface, and wherein the microcontroller is configured to monitor skin impedance at the electrode-skin interface and to deactivate the heating element when the monitored skin impedance falls below a threshold value indicating sufficient moisture for transcutaneous delivery of electrical stimulation.
Example 10 according to any one or more of examples 7 to 9, wherein the heating element comprises a thin-film resistive trace deposited or printed on a flexible substrate, the flexible substrate being positioned between the at least one stimulation electrode and the surface layer such that thermal energy from the resistive trace conducts through the surface layer to the plantar surface of the user's foot.
Example 11 according to any one or more of examples 1 to 10, further comprising one or more additional stimulation electrodes embedded within the multi-layer body at one or more anatomical zones of the foot in addition to the arch region and the heel region, wherein the stimulation circuit is configured to selectively activate individual electrodes or combinations of electrodes to direct stimulation to targeted plantar regions.
Example 12 according to any one or more of examples 1 to 11, wherein the surface layer and the cushioning layer extend anteriorly beyond the support layer and the base structure at a toe region of the insole to define a trimming zone configured to be cut to adjust the length of the insole to fit within footwear of a selected size.
Example 13 according to any one or more of examples 1 to 12, wherein the stimulation circuit is configured to deliver electrical current not exceeding 80 milliamps peak through the at least two stimulation electrodes.
Example 14 according to any one or more of examples 1 to 13, wherein the conductive fabric patches comprise silver-carbon fibers woven or knitted into a textile structure, the silver-carbon fibers providing both electrical conductivity and moisture absorption capacity at the electrode-skin interface.
Example 15 according to any one or more of examples 1 to 14, wherein the at least two stimulation electrodes are coated with a conductive polymer comprising poly(3,4-ethylenedioxythiophene) polystyrene sulfonate deposited on an electrode substrate and thermally cured to form a mechanically stable conductive film, the conductive polymer coating reducing interfacial impedance at the electrode-skin interface by providing mixed ionic and electronic conductivity at the electrode surface.
Example 16 according to any one or more of examples 1 to 15, wherein the stimulation circuit further comprises one or more pressure sensors disposed within the multi-layer body and configured to generate a pressure signal representative of a force applied to the insole by the user's foot, and wherein the microcontroller is configured to receive the pressure signal, determine based on the pressure signal whether the user's foot is in a stationary state or an ambulatory state, and deactivate the stimulation circuit in response to determining that the user's foot is in the ambulatory state.
Example 17 according to example 16, wherein the microcontroller is configured to reactivate the stimulation circuit in response to determining that the user's foot has returned to the stationary state.
Example 18 according to any one or more of examples 16 to 17, wherein the microcontroller determines the ambulatory state based on at least one of a periodic variation in the pressure signal consistent with a gait cycle, a rate of change of the pressure signal exceeding a predefined threshold, or a differential pressure reading between two or more pressure sensors positioned at different locations along the insole body.
Example 19 is a method of manufacturing a therapeutic insole, the method comprising forming a base structure from a flexible polymer material, the base structure defining a plantar support surface shaped to conform to an underside of a human foot and including an enclosure cavity at a heel portion of the base structure, assembling a support layer on the base structure, the support layer comprising a molded elastomeric material, forming one or more wire routing channels within the support layer extending from the enclosure cavity toward an arch region and a heel region of the support layer, positioning at least two stimulation electrodes within the multi-layer body, a first electrode at a position corresponding to the arch region and a second electrode at a position corresponding to the heel region, assembling a cushioning layer on the support layer and a surface layer on the cushioning layer such that the at least two stimulation electrodes are accessible at or through the surface layer, routing a wiring harness through the one or more wire routing channels to electrically connect the at least two stimulation electrodes to a stimulation circuit, and installing the stimulation circuit within the enclosure cavity, the stimulation circuit comprising a microcontroller and one or more power sources configured to deliver electrical stimulation through the at least two stimulation electrodes.
Example 20 according to example 19, wherein forming the support layer comprises casting a liquid elastomeric material into a mold defining the plantar support surface contour and the one or more wire routing channels, and curing the elastomeric material to form a unitary support layer having the wire routing channels integrally formed therein.
Example 21 according to any one or more of examples 19 to 20, wherein installing the stimulation circuit further comprises securing a lid over the enclosure cavity, the lid formed from a polymer material selected for a flexural modulus that resists deformation into contact with the stimulation circuit under a compressive load from a user's body weight, and positioning one or more internal support structures within the enclosure cavity between the lid and the stimulation circuit to distribute compressive loads away from the stimulation circuit components.
Example 22 according to any one or more of examples 19 to 21, further comprising securing conductive fabric patches to the surface layer at positions aligned with the at least two stimulation electrodes, the conductive fabric patches formed from a moisture-retaining conductive material electrically coupled to the at least two stimulation electrodes through the surface layer.
Example 23 according to any one or more of examples 19 to 22, further comprising positioning a heating element within the multi-layer body adjacent to at least one of the at least two stimulation electrodes, the heating element configured to apply localized thermal energy to a plantar surface of a user's foot to induce perspiration at an electrode-skin interface, and electrically connecting the heating element to the stimulation circuit within the enclosure cavity such that the microcontroller is configured to regulate activation of the heating element.
Example 24 according to any one or more of examples 19 to 23, further comprising applying a conductive polymer coating to the at least two stimulation electrodes prior to positioning the electrodes within the multi-layer body, the conductive polymer coating comprising poly(3,4-ethylenedioxythiophene) polystyrene sulfonate deposited from an aqueous or solvent-based dispersion and thermally cured to form a mechanically stable conductive film on the electrode surfaces.
Example 25 according to any one or more of examples 19 to 24, wherein the surface layer and the cushioning layer are formed to extend anteriorly beyond the support layer and the base structure at a toe region of the insole to define a trimming zone configured to be cut to adjust the length of the insole to fit within footwear of a selected size.
Example 26 is a therapeutic stimulation system comprising a therapeutic insole configured to be inserted into footwear, the insole comprising a base structure defining a plantar support surface shaped to conform to an underside of a human foot, a multi-layer body comprising a support layer, a cushioning layer disposed on the support layer, and a surface layer disposed on the cushioning layer, at least two stimulation electrodes embedded within the multi-layer body, a first electrode positioned at a location corresponding to an arch region of the foot and a second electrode positioned at a location corresponding to a heel region of the foot, an electronics enclosure integrated into a heel portion of the base structure, the electronics enclosure housing a stimulation circuit comprising a microcontroller, one or more power sources, and a wireless communication module, and one or more wire routing channels formed within the multi-layer body and extending from the electronics enclosure to each of the at least two stimulation electrodes, and an external device executing a software application configured to transmit stimulation control signals to the wireless communication module, wherein the stimulation circuit is configured to generate a pulsed electrical stimulation signal and deliver the signal through the at least two stimulation electrodes in response to stimulation control signals received from the external device.
Example 27 according to example 26, further comprising a conductive foot covering configured to be worn over a user's foot between the user's foot and the insole, the conductive foot covering comprising conductive regions formed from conductive yarn or conductive fabric positioned to align with the at least two stimulation electrodes when the foot covering is worn, wherein the conductive regions provide an electrical conduction path between the at least two stimulation electrodes and the plantar surface of the user's foot.
Example 28 according to any one or more of examples 26 to 27, wherein the stimulation control signals specify at least one of stimulation intensity level, stimulation pulse frequency, or stimulation on and off state, and wherein the microcontroller adjusts the pulsed electrical stimulation signal delivered through the at least two stimulation electrodes in response to the received stimulation control signals.
Example 29 according to any one or more of examples 26 to 28, wherein the electronics enclosure comprises an enclosure body and a lid secured over the enclosure body, the enclosure body including one or more internal support structures positioned between the lid and the stimulation circuit, the internal support structures configured to prevent contact between the lid and the stimulation circuit when the insole is subjected to a compressive load from a user's body weight.
Example 30 according to any one or more of examples 26 to 29, wherein the stimulation circuit further comprises at least one light-emitting indicator configured to convey to a user one or more device states comprising at least one of power on state, low battery state, wireless pairing mode, and wireless connection established state.
Example 31 according to any one or more of examples 26 to 30, wherein the stimulation circuit further comprises a heating element positioned adjacent to at least one of the at least two stimulation electrodes, the heating element configured to apply localized thermal energy to induce perspiration at an electrode-skin interface to reduce skin impedance, and wherein the microcontroller is configured to activate the heating element in response to a control signal received from the external device via the wireless communication module or in response to a detected high-impedance condition at the electrode-skin interface.
Example 32 according to any one or more of examples 26 to 31, wherein the software application is further configured to display a pressure distribution map of the user's foot based on pressure sensor data received from the therapeutic insole via the wireless communication module, and to transmit adjusted stimulation control signals based on the displayed pressure distribution.
Example 33 according to any one or more of examples 26 to 32, wherein the stimulation circuit further comprises one or more pressure sensors disposed within the multi-layer body, and wherein the microcontroller is configured to determine based on pressure sensor data whether the user's foot is in a stationary state or an ambulatory state and to suspend the pulsed electrical stimulation signal upon detecting the ambulatory state.
Example 34 according to any one or more of examples 26 to 33, wherein the at least two stimulation electrodes are coated with a conductive polymer comprising poly(3,4-ethylenedioxythiophene) polystyrene sulfonate, the conductive polymer coating reducing interfacial impedance at the electrode-skin interface.

In view of the above-described implementations of subject matter this application discloses the following list of examples, wherein one feature of an example in isolation or more than one feature of an example, taken in combination and, optionally, in combination with one or more features of one or more further examples are further examples also falling within the disclosure of this application.
Example 35 is an electrical stimulation system for a foot, the system comprising an insole configured to be inserted into footwear, the insole having an upper surface and one or more stimulation electrodes embedded on or accessible through the upper surface at predetermined stimulation zones corresponding to anatomical regions of a plantar surface of a human foot, and a conductive foot covering configured to be worn over a user's foot between the user's foot and the insole, the conductive foot covering comprising one or more conductive regions formed from conductive yarn or conductive fabric, the one or more conductive regions being positioned on a plantar-facing surface of the conductive foot covering to align with the predetermined stimulation zones of the insole when the conductive foot covering is worn on the user's foot and the user's foot is placed on the insole, wherein the one or more conductive regions provide an electrical conduction path between the one or more stimulation electrodes and the plantar surface of the user's foot, and wherein the conductive fabric of the one or more conductive regions is configured to retain moisture at an interface between the conductive foot covering and the plantar surface of the user's foot to sustain delivery of electrical stimulation under low-perspiration conditions.
Example 36 according to example 35, wherein the predetermined stimulation zones comprise a first stimulation zone corresponding to an arch region of the foot and a second stimulation zone corresponding to a heel region of the foot, and wherein the one or more conductive regions comprise a first conductive region positioned on the conductive foot covering to align with the first stimulation zone and a second conductive region positioned on the conductive foot covering to align with the second stimulation zone.
Example 37 according to any one or more of examples 35 to 36, wherein the conductive yarn or conductive fabric of the one or more conductive regions comprises silver-carbon fibers woven or knitted into a textile structure, the silver-carbon fibers providing both electrical conductivity and moisture absorption capacity.
Example 38 according to any one or more of examples 35 to 37, wherein the conductive yarn or conductive fabric of the one or more conductive regions comprises silver-coated nylon fibers knitted into the textile structure of the conductive foot covering.
Example 39 according to any one or more of examples 35 to 38, wherein the conductive yarn or conductive fabric of the one or more conductive regions comprises stainless steel filament yarns integrated into the textile structure of the conductive foot covering.
Example 40 according to any one or more of examples 35 to 39, wherein the conductive foot covering is shaped as a sock configured to cover the user's foot from the toes to at least the ankle, and wherein non-conductive textile regions of the sock surround the one or more conductive regions such that the conductive regions are spatially isolated from each other on the plantar-facing surface of the sock.
Example 41 according to any one or more of examples 35 to 40, wherein the conductive foot covering is shaped as a partial foot wrap or sleeve configured to cover a portion of the plantar surface of the user's foot corresponding to the predetermined stimulation zones without covering the entirety of the user's foot.
Example 42 according to any one or more of examples 35 to 41, wherein each of the one or more conductive regions has a shape and area that matches or exceeds a shape and area of a corresponding stimulation electrode on the upper surface of the insole, such that when the conductive foot covering is worn and the user's foot is placed on the insole, the conductive region fully overlaps the corresponding stimulation electrode.
Example 43 according to any one or more of examples 35 to 42, wherein the conductive foot covering further comprises one or more registration features configured to assist alignment of the one or more conductive regions with the predetermined stimulation zones, the one or more registration features comprising at least one of tactile indicators perceptible by the user's foot, visual alignment markings on the conductive foot covering, or geometric contouring of the conductive foot covering shaped to conform to an anatomical contour of the user's foot and thereby guide the conductive regions into alignment with the stimulation zones.
Example 44 according to any one or more of examples 35 to 43, wherein the insole further comprises an electronics enclosure integrated into a heel portion of the insole, the electronics enclosure housing a stimulation circuit comprising a microcontroller and one or more power sources, and one or more wire routing channels formed within the insole extending from the electronics enclosure to each of the one or more stimulation electrodes, and wherein the stimulation circuit is configured to deliver transcutaneous electrical nerve stimulation through the one or more stimulation electrodes and through the one or more conductive regions of the conductive foot covering to the plantar surface of the user's foot.
Example 45 according to any one or more of examples 35 to 44, wherein the insole comprises a multi-layer body disposed on a base structure, the multi-layer body comprising a support layer, a cushioning layer disposed on the support layer, and a surface layer disposed on the cushioning layer, and wherein the one or more stimulation electrodes are embedded within the multi-layer body and are accessible at or through the surface layer.
Example 46 according to any one or more of examples 35 to 45, wherein the stimulation circuit further comprises a wireless communication module configured to receive stimulation control signals from an external device executing a software application, and wherein the stimulation circuit is configured to adjust at least one of stimulation intensity, stimulation pulse frequency, or stimulation on and off state in response to the received stimulation control signals.
Example 47 according to any one or more of examples 35 to 46, wherein the insole further comprises a heating element positioned adjacent to at least one of the one or more stimulation electrodes, the heating element configured to apply localized thermal energy to the plantar surface of the user's foot through the conductive foot covering to induce perspiration at an interface between the conductive foot covering and the user's skin, thereby reducing skin impedance and improving transcutaneous delivery of electrical stimulation through the one or more conductive regions.
Example 48 according to any one or more of examples 35 to 47, wherein the one or more stimulation electrodes are coated with a conductive polymer comprising poly(3,4-ethylenedioxythiophene) polystyrene sulfonate deposited on an electrode substrate and thermally cured to form a mechanically stable conductive film, the conductive polymer coating reducing interfacial impedance at the electrode surface.
Example 49 according to any one or more of examples 35 to 48, wherein the conductive fabric of the one or more conductive regions has a sheet resistance of less than 10 ohms per square, providing a low-impedance electrical path between the stimulation electrodes and the user's skin.
Example 50 according to any one or more of examples 35 to 49, wherein the conductive foot covering is washable and the one or more conductive regions maintain electrical conductivity after at least 50 wash cycles.
Example 51 according to any one or more of examples 35 to 50, wherein the system further comprises one or more pressure sensors disposed within the insole and configured to generate a pressure signal representative of a force applied to the insole by the user's foot, and wherein a microcontroller within the insole is configured to receive the pressure signal, determine based on the pressure signal whether the user's foot is in a stationary state or an ambulatory state, and deactivate the stimulation circuit in response to determining that the user's foot is in the ambulatory state.
Example 52 is a method of delivering electrical stimulation to a plantar surface of a user's foot, the method comprising providing an insole configured to be inserted into footwear, the insole having one or more stimulation electrodes embedded on or accessible through an upper surface of the insole at predetermined stimulation zones corresponding to anatomical regions of the plantar surface, placing a conductive foot covering over the user's foot, the conductive foot covering comprising one or more conductive regions formed from conductive yarn or conductive fabric positioned to align with the predetermined stimulation zones when the conductive foot covering is worn, inserting the user's foot with the conductive foot covering into the footwear containing the insole such that the one or more conductive regions contact the one or more stimulation electrodes and establish an electrical conduction path between the stimulation electrodes and the plantar surface of the user's foot, and activating a stimulation circuit within the insole to deliver electrical stimulation through the one or more stimulation electrodes and through the one or more conductive regions to the plantar surface of the user's foot.
Example 53 according to example 52, wherein the conductive fabric of the one or more conductive regions retains moisture at the interface between the conductive foot covering and the plantar surface of the user's foot, and wherein the retained moisture sustains delivery of electrical stimulation through the conductive regions under low-perspiration conditions.
Example 54 according to any one or more of examples 52 to 53, further comprising activating a heating element within the insole positioned adjacent to at least one of the one or more stimulation electrodes to apply localized thermal energy through the conductive foot covering to the plantar surface of the user's foot, thereby inducing perspiration at the interface between the conductive foot covering and the user's skin and reducing skin impedance prior to or during delivery of the transcutaneous electrical nerve stimulation.
Example 55 according to any one or more of examples 52 to 54, further comprising transmitting stimulation control signals from an external device executing a software application to a wireless communication module within the insole, the stimulation control signals specifying at least one of stimulation intensity level, stimulation pulse frequency, or stimulation on and off state, and adjusting the transcutaneous electrical nerve stimulation delivered through the one or more stimulation electrodes in response to the received stimulation control signals.
Example 56 according to any one or more of examples 52 to 55, further comprising detecting, via one or more pressure sensors within the insole, a transition of the user's foot from a stationary state to an ambulatory state, and automatically deactivating the stimulation circuit in response to detecting the transition to the ambulatory state.
Example 57 is a conductive foot covering for use with a transcutaneous electrical nerve stimulation insole, the conductive foot covering comprising a textile body shaped to be worn over a user's foot, and one or more conductive regions disposed on a plantar-facing surface of the textile body, the one or more conductive regions formed from conductive yarn or conductive fabric and positioned at locations corresponding to predetermined stimulation zones of a transcutaneous electrical nerve stimulation insole, wherein the one or more conductive regions are configured to provide an electrical conduction path between stimulation electrodes of the insole and a plantar surface of the user's foot when the conductive foot covering is worn and the user's foot is placed on the insole, and wherein the conductive fabric of the one or more conductive regions is configured to retain moisture at the interface between the conductive foot covering and the plantar surface of the user's foot to sustain delivery of electrical stimulation under low-perspiration conditions.
Example 58 according to example 57, wherein the one or more conductive regions comprise a first conductive region positioned at a location corresponding to an arch region of the user's foot and a second conductive region positioned at a location corresponding to a heel region of the user's foot.
Example 59 according to any one or more of examples 57 to 58, wherein the conductive yarn or conductive fabric comprises silver-carbon fibers providing both electrical conductivity and moisture absorption capacity.
Example 60 according to any one or more of examples 57 to 59, wherein non-conductive textile regions of the textile body surround and spatially isolate the one or more conductive regions from each other on the plantar-facing surface to maintain separate electrical paths for each conductive region.
Example 61 according to any one or more of examples 57 to 60, wherein the textile body further comprises one or more registration features configured to assist alignment of the one or more conductive regions with stimulation electrodes of the insole, the registration features comprising at least one of tactile indicators, visual alignment markings, or geometric contouring shaped to conform to an anatomical contour of the user's foot.
Example 62 according to any one or more of examples 57 to 61, wherein the conductive foot covering is washable and the one or more conductive regions maintain electrical conductivity after at least 50 wash cycles.

In view of the above-described implementations of subject matter this application discloses the following list of examples, wherein one feature of an example in isolation or more than one feature of an example, taken in combination and, optionally, in combination with one or more features of one or more further examples are further examples also falling within the disclosure of this application.
Example 63 is an insole configured to be placed inside footwear, the insole comprising a multi-layer body including a structural support layer, a cushioning layer disposed on the structural support layer, and a surface layer disposed on the cushioning layer, at least two stimulation electrodes embedded within or accessible through the surface layer, a first stimulation electrode positioned at a location corresponding to an arch region of a human foot and a second stimulation electrode positioned at a location corresponding to a heel region of the human foot, an electronics enclosure integrated into the insole, the electronics enclosure housing a printed circuit board, one or more batteries electrically connected to the printed circuit board, and a microcontroller mounted on the printed circuit board, wherein the microcontroller is configured to generate a pulse-width-modulated signal, and wherein an operational amplifier circuit mounted on the printed circuit board is configured to receive the pulse-width-modulated signal from the microcontroller and to invert and offset the pulse-width-modulated signal to produce a biphasic symmetric stimulation waveform, the biphasic symmetric stimulation waveform being delivered through the at least two stimulation electrodes such that electrical current flows in alternating polarity between the first stimulation electrode and the second stimulation electrode, and wherein the insole provides both passive structural support for a user's foot through the structural support layer and active transcutaneous electrical nerve stimulation through the at least two stimulation electrodes.
Example 64 according to example 63, wherein the biphasic symmetric stimulation waveform comprises a positive pulse phase and a negative pulse phase of substantially equal amplitude and duration relative to a zero-baseline reference, such that a net electrical charge delivered through the at least two stimulation electrodes over one complete cycle of the waveform is substantially zero, thereby preventing accumulation of ions at the electrode-skin interface.
Example 65 according to any one or more of examples 63 to 64, wherein the microcontroller is configured to control the pulse-width-modulated signal by adjusting at least one of a pulse frequency, a duty cycle, or a number of samples per pulse cycle, and wherein the operational amplifier circuit inverts and offsets the pulse-width-modulated signal for each combination of adjusted parameters to maintain biphasic symmetry across a range of stimulation intensities.
Example 66 according to any one or more of examples 63 to 65, wherein the operational amplifier circuit comprises a single operational amplifier configured in an inverting amplifier topology with a DC offset voltage applied to a non-inverting input of the operational amplifier, the DC offset voltage being derived from a voltage divider connected between a supply rail of the one or more batteries and a circuit ground, such that the output of the operational amplifier swings symmetrically above and below the DC offset voltage in response to the pulse-width-modulated signal from the microcontroller.
Example 67 according to any one or more of examples 63 to 66, wherein the one or more batteries comprise two batteries connected in series to provide a combined supply voltage, and wherein a center tap between the two batteries defines a virtual ground reference for the biphasic symmetric stimulation waveform, such that the positive pulse phase is referenced above the virtual ground and the negative pulse phase is referenced below the virtual ground.
Example 68 according to any one or more of examples 63 to 67, wherein the one or more batteries comprise a single battery, and wherein the printed circuit board further comprises an H-bridge switching circuit comprising four switching elements arranged in a bridge topology between the single battery and the at least two stimulation electrodes, the microcontroller being configured to drive the four switching elements in alternating diagonal pairs to reverse the polarity of voltage applied across the at least two stimulation electrodes during each phase of the biphasic symmetric stimulation waveform.
Example 69 according to example 68, wherein the printed circuit board further comprises a DC-DC converter positioned between the single battery and the H-bridge switching circuit, the DC-DC converter configured to step up or regulate the output voltage of the single battery to a level sufficient to drive the stimulation current through skin impedance at the electrode-skin interface while remaining within safe current limits for transcutaneous electrical nerve stimulation.
Example 70 according to any one or more of examples 63 to 69, wherein the stimulation circuit is configured to operate in a current-controlled mode in which a current-sensing element is positioned in an output path between the operational amplifier circuit and the at least two stimulation electrodes, and wherein the microcontroller monitors a voltage across the current-sensing element and adjusts an output voltage of the operational amplifier circuit to maintain a target current setpoint through the at least two stimulation electrodes despite fluctuations in skin impedance at the electrode-skin interface.
Example 71 according to example 70, wherein the microcontroller enforces a maximum voltage compliance limit on the output of the operational amplifier circuit to prevent the output voltage from exceeding a safe operating range, and wherein the target current setpoint is adjustable via a wireless communication module or a mobile application communicatively coupled to the microcontroller.
Example 72 according to any one or more of examples 63 to 71, wherein the electronics enclosure comprises an enclosure body and a lid secured over the enclosure body, and wherein one or more internal support structures are positioned within the enclosure body between the lid and the printed circuit board and between the lid and the one or more batteries, the one or more internal support structures being configured to transfer a compressive load applied to the lid directly to a floor of the enclosure body without transmitting the load to the printed circuit board or the one or more batteries.
Example 73 according to example 72, wherein the lid is formed from polyethylene terephthalate glycol or polylactic acid having a flexural modulus sufficient to limit deflection of the lid to less than a dimension of a gap between an underside of the lid and an uppermost surface of the one or more batteries when the insole is subjected to a distributed compressive load of at least 1112 Newtons.
Example 74 according to any one or more of examples 63 to 73, wherein the structural support layer comprises a molded silicone elastomer, the cushioning layer comprises at least one of ethylene vinyl acetate foam or a polyurethane-based open-cell foam, and the insole further comprises a base structure formed from thermoplastic polyurethane or polyethylene terephthalate glycol disposed beneath the structural support layer, the base structure defining a plantar support surface shaped to conform to the underside of the human foot.
Example 75 according to any one or more of examples 63 to 74, wherein one or more wire routing channels are formed within the structural support layer, the wire routing channels extending from the electronics enclosure to each of the at least two stimulation electrodes, and wherein a wiring harness is routed through the wire routing channels to electrically connect the printed circuit board to the first stimulation electrode and the second stimulation electrode.
Example 76 according to example 75, wherein the wire routing channels are enclosed channels integrally formed within the structural support layer during a molding operation, the channels extending longitudinally from the electronics enclosure along a medial side of the insole toward the arch region and diverging to route separate conductive pathways to the first stimulation electrode and the second stimulation electrode.
Example 77 according to any one or more of examples 63 to 76, further comprising conductive fabric patches disposed on the surface layer at positions aligned with the at least two stimulation electrodes, the conductive fabric patches formed from a moisture-retaining conductive material configured to provide an electrical conduction path between the at least two stimulation electrodes and a plantar surface of the user's foot and to retain moisture at the electrode-skin interface to sustain delivery of electrical stimulation under low-perspiration conditions.
Example 78 according to any one or more of examples 63 to 77, wherein the at least two stimulation electrodes are coated with a conductive polymer comprising poly(3,4-ethylenedioxythiophene) polystyrene sulfonate deposited on an electrode substrate and thermally cured to form a mechanically stable conductive film, the conductive polymer coating reducing interfacial impedance at the electrode-skin interface by providing mixed ionic and electronic conductivity at the electrode surface.
Example 79 according to any one or more of examples 63 to 78, further comprising a heating element positioned adjacent to at least one of the at least two stimulation electrodes, the heating element configured to apply localized thermal energy to induce perspiration at the electrode-skin interface, thereby reducing skin impedance and improving transcutaneous delivery of electrical stimulation through the at least two stimulation electrodes.
Example 80 according to any one or more of examples 63 to 79, further comprising one or more additional stimulation electrodes embedded within the multi-layer body at one or more anatomical zones of the foot in addition to the arch region and the heel region, wherein the microcontroller is configured to selectively activate individual electrodes or combinations of electrodes to direct stimulation to targeted plantar regions.
Example 81 according to any one or more of examples 63 to 80, wherein the at least two stimulation electrodes are arranged in a guard ring electrode configuration in which a central active electrode is concentrically surrounded by an annular return electrode, the annular return electrode being configured to intercept laterally spreading current at a periphery of the central active electrode and conduct the current back through a return path of the stimulation circuit, thereby confining the delivered current field to a localized volume of tissue beneath the central active electrode.
Example 82 according to any one or more of examples 63 to 81, wherein the at least two stimulation electrodes are arranged in a tripolar or quadripolar current steering configuration comprising three or four electrodes positioned in a spatial arrangement within a single stimulation pad region, and wherein the microcontroller independently controls an amplitude of the stimulation signal applied to each electrode in the configuration to electronically steer a resulting current field along a desired spatial path within tissue beneath the stimulation pad without physically repositioning the electrodes.
Example 83 according to any one or more of examples 63 to 82, further comprising a wireless communication module mounted on the printed circuit board and configured to communicate with an external device executing a software application, wherein the software application is configured to transmit stimulation control signals to the wireless communication module specifying at least one of stimulation intensity level, stimulation pulse frequency, or stimulation on and off state, and wherein the microcontroller adjusts the biphasic symmetric stimulation waveform delivered through the at least two stimulation electrodes in response to the received stimulation control signals.
Example 84 according to any one or more of examples 63 to 83, further comprising one or more pressure sensors disposed within the multi-layer body and configured to generate a pressure signal representative of a force applied to the insole by the user's foot, and wherein the microcontroller is configured to receive the pressure signal, determine based on the pressure signal whether the user's foot is in a stationary state or an ambulatory state, and deactivate the stimulation circuit in response to determining that the user's foot is in the ambulatory state.
Example 85 according to any one or more of examples 63 to 84, wherein the printed circuit board further comprises an overcurrent protection device positioned in a power supply path between the one or more batteries and the operational amplifier circuit, the overcurrent protection device configured to interrupt current flow when a current through the power supply path exceeds a predetermined threshold.
Example 86 according to any one or more of examples 63 to 85, wherein the stimulation circuit is configured to deliver electrical current not exceeding 80 milliamps peak through the at least two stimulation electrodes at pulse parameters within ranges established for safe transcutaneous electrical nerve stimulation of human tissue.
Example 87 according to any one or more of examples 63 to 86, wherein the surface layer and the cushioning layer extend anteriorly beyond the structural support layer at a toe region of the insole to define a trimming zone configured to be cut to adjust the length of the insole to fit within footwear of a selected size.
Example 88 according to any one or more of examples 63 to 87, further comprising at least one light-emitting indicator mounted on the printed circuit board and configured to convey to the user one or more device states comprising at least one of power on state, low battery state, wireless pairing mode, and wireless connection established state.
Example 89 according to any one or more of examples 63 to 88, wherein the printed circuit board is a flexible printed circuit board comprising a polyimide substrate, the flexible printed circuit board being configured to conform to a contour of a floor of the electronics enclosure to reduce a vertical profile of the electronics enclosure within the insole.
Example 90 according to any one or more of examples 63 to 89, wherein the printed circuit board is a rigid-flex hybrid construction comprising rigid board sections carrying the microcontroller and the operational amplifier circuit joined by flexible polyimide bridge sections, the flexible bridge sections accommodating bending between the rigid sections and allowing the printed circuit board assembly to conform to an internal geometry of the electronics enclosure.
Example 91 is a method of providing electrical stimulation to a foot of a user suffering from pain associated with flat feet or fallen arches, the method comprising inserting into footwear an insole comprising a multi-layer body providing passive structural support for the user's foot through a structural support layer and a cushioning layer, and at least two stimulation electrodes embedded within the multi-layer body, a first stimulation electrode positioned at a location corresponding to an arch region of the foot and a second stimulation electrode positioned at a location corresponding to a heel region of the foot, generating, by a microcontroller mounted on a printed circuit board housed within an electronics enclosure integrated into the insole, a pulse-width-modulated signal, inverting and offsetting, by an operational amplifier circuit mounted on the printed circuit board, the pulse-width-modulated signal to produce a biphasic symmetric stimulation waveform, and delivering the biphasic symmetric stimulation waveform through the at least two stimulation electrodes such that electrical current flows in alternating polarity between the first stimulation electrode and the second stimulation electrode to provide electrical stimulation to the plantar surface of the user's foot.
Example 92 according to example 91, wherein the biphasic symmetric stimulation waveform has a net-zero charge over each complete cycle, thereby preventing accumulation of ions at the electrode-skin interface during delivery of the transcutaneous electrical nerve stimulation.
Example 93 according to any one or more of examples 91 to 92, further comprising receiving, by the microcontroller via a wireless communication module, stimulation control signals from an external device executing a software application, and adjusting at least one of a pulse frequency, a duty cycle, or an intensity of the biphasic symmetric stimulation waveform in response to the received stimulation control signals.
Example 94 according to any one or more of examples 91 to 93, further comprising detecting, by the microcontroller via one or more pressure sensors disposed within the multi-layer body, a transition of the user's foot from a stationary state to an ambulatory state, and automatically suspending delivery of the biphasic symmetric stimulation waveform through the at least two stimulation electrodes in response to detecting the transition to the ambulatory state.
Example 95 according to any one or more of examples 91 to 94, further comprising activating a heating element positioned adjacent to at least one of the at least two stimulation electrodes to apply localized thermal energy to the plantar surface of the user's foot to induce perspiration at the electrode-skin interface prior to delivering the biphasic symmetric stimulation waveform, thereby reducing skin impedance and improving transcutaneous delivery of electrical stimulation.

1. A therapeutic insole configured to be inserted into footwear, comprising:
a base structure defining a plantar support surface shaped to conform to an underside of a human foot;
a multi-layer body disposed on the base structure, the multi-layer body comprising a support layer, a cushioning layer disposed on the support layer, and a surface layer disposed on the cushioning layer;
at least two stimulation electrodes embedded within the multi-layer body, a first electrode positioned at a location corresponding to an arch region of the foot and a second electrode positioned at a location corresponding to a heel region of the foot, wherein the at least two stimulation electrodes are accessible at or through the surface layer;
an electronics enclosure integrated into the base structure at a heel portion thereof, the electronics enclosure housing a stimulation circuit comprising a microcontroller and one or more power sources; and
one or more wire routing channels formed within the multi-layer body and extending from the electronics enclosure to each of the at least two stimulation electrodes;
wherein the stimulation circuit is configured to deliver electrical stimulation through the at least two stimulation electrodes.

2. The therapeutic insole of claim 1, wherein the stimulation circuit further comprises an operational amplifier configured to receive a pulse-width-modulated signal from the microcontroller and to invert and offset the pulse-width-modulated signal to produce a biphasic symmetric stimulation waveform delivered through the at least two stimulation electrodes such that electrical current flows in alternating polarity between the first electrode and the second electrode.

3. The therapeutic insole of claim 1, wherein the electronics enclosure comprises an enclosure body and a lid secured over the enclosure body, the lid formed from a material having a flexural modulus sufficient to resist deformation into contact with components of the stimulation circuit when the insole is subjected to a distributed compressive load corresponding to a body weight of a user, and wherein the enclosure body includes one or more internal support structures positioned between the lid and the stimulation circuit to distribute compressive loads away from the stimulation circuit components.

4. The therapeutic insole of claim 1, wherein the support layer comprises a molded elastomeric material, the cushioning layer comprises at least one of ethylene vinyl acetate foam or an open-cell polyurethane foam, and the base structure comprises a thermoplastic polyurethane.

5. The therapeutic insole of claim 1, wherein the one or more wire routing channels are enclosed channels formed within the support layer and extend longitudinally from the electronics enclosure toward the arch region, routing separate conductive pathways to each of the at least two stimulation electrodes.

6. The therapeutic insole of claim 1, further comprising conductive fabric patches disposed on the surface layer at positions aligned with the at least two stimulation electrodes, the conductive fabric patches formed from a moisture-retaining conductive material configured to provide an electrical conduction path between the at least two stimulation electrodes and a plantar surface of a foot of a user and to retain moisture at an electrode-skin interface to sustain delivery of electrical stimulation under low-perspiration conditions.

7. The therapeutic insole of claim 1, wherein the stimulation circuit further comprises a heating element positioned adjacent to at least one of the at least two stimulation electrodes, the heating clement configured to apply localized thermal energy to induce perspiration at an electrode-skin interface, thereby reducing skin impedance and improving transcutaneous delivery of electrical stimulation through the at least two stimulation electrodes.

8. The therapeutic insole of claim 1, further comprising one or more additional stimulation electrodes embedded within the multi-layer body at one or more anatomical zones of the foot in addition to the arch region and the heel region, wherein the stimulation circuit is configured to selectively activate individual electrodes or combinations of electrodes to direct stimulation to targeted plantar regions.

9. The therapeutic insole of claim 1, wherein the surface layer and the cushioning layer extend anteriorly beyond the support layer and the base structure at a toe region of the insole to define a trimming zone configured to be cut to adjust the length of the insole to fit within footwear of a selected size.

10. The therapeutic insole of claim 1, wherein the stimulation circuit is configured to deliver electrical current not exceeding 80 milliamps peak through the at least two stimulation electrodes.

11-20. (canceled)
